SEQUENCE LISTING (2 sequences)

SEQ ID NO: 1 - protein, 300 aa, unidentified
MCARGQVGRGTQLRTGRPCSQVPGSRWRPERLLRRQRAGGRPSRPHPARARPGLSLPATLLGSRAAAAVPLPLPPALAPGDPAMPVRTECPPPAGASAASAASLIPPPPINTQQPGVATSLLYSGSKFRGHQKSKGNSYDVEVVLQHVDTGNSYLCGYLKIKGLTEEYPTLTTFFEGEIISKKHPFLTRKWDADEDVDRKHWGKFLAFYQYAKSFNSDDFDYEELKNGDYVFMRWKEQFLVPDHTIKDISGASFAGFYYICFQKSAASIEGYYYHRSSEWYQSLNLTHVPEHSAPIYEFR

SEQ ID NO: 2 - protein, 299 aa, unidentified
MCARGQVGRGTQLRTGRPCSQVPGSRWRPERLLRRQRAGGRPSRPHPARARPGLSLPATLLGSRAAAAVPLPLPPALAPGDPAMPVRTECPPPAGASAASAASLIPPPPINTQQPGVATSLLYSGSKFRGHQKSKGNSYDVEVVLQHVDTGNSYLCGYLKIKGLTEEYPTLTTFFEGEIISKKHPFLTRKWDADEDVDRKHWGKFLAFYQYAKSFNSDDFDYEELKNGDYVFMRWKEQFLVPDHTIKDISGXXFAGFYYICFQKSAASIEGYYYHRSSEWYQXNLTHVPEHSAPIYEFR

(19) United States
(12) Patent Application Publication
(10) Pub. No.: US 2025/0026797 A1
(43) Pub. Date: Jan. 23, 2025

(54) GID4 N-TERMINAL PROLINE BINDER AND DETERMINING A SEQUENCE OF A PEPTIDE

(71) Applicant: Government of the United States of America, as represented by the Secretary of Commerce, Gaithersburg, MD (US)

(72) Inventors: Zvi Kelman, Gaithersburg, MD (US); John P. Marino, Silver Spring, MD (US); Svetlana Pavlova Ikonomova, Rockville, MD (US)

(21) Appl. No.: 18/903,043

(22) Filed: Oct. 1, 2024

Related U.S. Application Data
(63) Continuation-in-part of application No. 17/837,082, filed on Jun. 10, 2022.
Application No. 17/837,082 is a continuation of application No. 16/986,334, filed on Aug. 6, 2020, now Pat. No. 11,390,653.
Application No. 16/986,334 is a division of application No. 16/395,407, filed on Apr. 26, 2019, now Pat. No. 10,836,798.
(60) Provisional application No. 62/757,271, filed on Nov. 8, 2018.
(60) Provisional application No. 63/654,539, filed on May 31, 2024.

(51) Int. Cl. C07K 14/195; C07K 1/13; G01N 33/58

(52) U.S. Cl. CPC C07K 14/195; C07K 1/13; G01N 33/582

(57) ABSTRACT

A GID4 N-terminal proline binder includes: a variant of a GID4 protein that includes a mutation, wherein the variant GID4 protein exhibits an increased binding affinity for an N-terminal proline residue as compared to a native GID4 protein.

### CROSS REFERENCE TO RELATED APPLICATIONS

This patent application is a continuation-in-part of U.S. patent application Ser. No. 17/837,082 (filed Jun. 10, 2022), which Is a continuation of U.S. patent application Ser. No. 16/986,334 (filed Aug. 6, 2020) that is now U.S. Pat. No. 11,390,653, which is a divisional of U.S. patent application Ser. No. 16/395,407 (filed Apr. 26, 2019) that is now U.S. Pat. No. 10,836,798, which claims benefit of U.S. Provisional Patent Application Ser. No. 62/757,271 (filed Nov. 8, 2018), and this patent application also claims the benefit of U.S. Provisional Patent Application Ser. No. 63/654,539 (filed May 31, 2024); all of the foregoing are incorporated herein by reference in their entireties.

### STATEMENT REGARDING FEDERALLY SPONSORED RESEARCH

This invention was made with United States Government support from the National Institute of Standards and Technology (NIST), an agency of the United States Department of Commerce. The Government has certain rights in this invention.

### REFERENCE TO AN ELECTRONIC SEQUENCE LISTING

The contents of the electronic sequence listing (18-066D1C1CP1.xml; Size: 4000 bytes; and date of creation: Sep. 27, 2024) is herein incorporated by reference in its entirety.

### BACKGROUND

The present invention generally relates to the field of protein sequencing, and more particularly to techniques for identifying N-terminal proline residues in peptides using engineered N-terminal amino acid binders.

Nucleic acid sequencing technologies have gone through extraordinary advancements in the past several decades, significantly increasing throughput while reducing cost. To create similar advancement in proteomics, numerous approaches are being investigated to advance protein sequencing. One of the promising approaches uses N-terminal amino acid binders (NAABs), also referred to as recognizers, that selectively can identify amino acids at the N-terminus of a peptide. However, there are only a few engineered NAABs currently available that bind to specific amino acids and meet the requirements of a biotechnology reagent. Therefore, additional NAABs need to be identified and engineered to enable confident identification and ultimately de novo protein sequencing.

Proline is a critical amino acid to identify in protein sequencing. The residue composes nearly 6% of human proteome and plays an important role in structural conformation of a protein by introducing a bend in the polypeptide chain. However, the side-chain cannot be chemically modified to allow recognition through attachment of fluorescent label or dye. A NAAB for proline would therefore be a valuable addition to the current NAAB repertoire and a worthy target for NAAB development.

A candidate protein for proline-binding NAAB exists in the eukaryotic N-degron system which targets proteins for degradation by the proteasome. The system recognizes an Nt-signal sequence or “degrons.” The GID4 protein is a part of the degradation machinery that recognizes the Nt-proline (Nt-Pro) and direct it for degradation. Based on structural studies, the depth and width of the binding pocket of GID4 results in the binding affinity and selectivity of the protein to not only be affected by the Nt-Pro, but also the residues that follow the Nt-Pro amino acid. The native GID4 protein has a weak affinity for N-terminal proline. Moreover, this binding is heavily impacted by the identity of the amino acids in the second and third position of the target peptide.

It is therefore an objective of the present invention to provide engineered variants of the GID4 protein that have an increased binding affinity for N-terminal proline residues and a reduced influence from residues in the second and third position of the target peptide when binding to N-terminal proline, thereby overcoming the above-mentioned disadvantages of the prior art at least in part.

Accordingly, methods and equipment for using engineered GID4 variants for the detection of N-terminal proline would be advantageous and would be favorably received in the art.

### BRIEF DESCRIPTION

One aspect of the present invention relates to a GID4 N-terminal proline binder. A GID4 N-terminal proline binder may be understood as a combination of two or more substances. It may be provided that the GID4 N-terminal proline binder comprises a variant of a GID4 protein. A GID4 protein can be understood as a protein that recognizes and binds to N-terminal proline residues in a peptide. This is particularly important for protein sequencing as proline residues cannot be readily chemically modified. The variant GID4 protein comprises at least one mutation selected from the group consisting of a substitution at position 252, position 253, and position 283 of SEQ ID NO: 1. One advantage of including a mutation selected from the group is that it provides the variant GID4 protein with a higher binding affinity for N-terminal proline residues as compared to the native protein. Moreover, the mutation can reduce the influence of the identity of the residue at the second and/or third position of the target peptide on the binding of the variant GID4 protein. The variant GID4 protein exhibits an increased binding affinity for an N-terminal proline residue as compared to a native GID4 protein. One advantage of an increased binding affinity is that it permits the variant GID4 protein to be more selective for peptides having an N-terminal proline.

It may be provided that the variant GID4 protein exhibits a reduced influence from the identity of a residue at the second position of a target peptide when binding to an N-terminal proline residue as compared to a native GID4 protein. One advantage of a reduced influence from the identity of the second residue is that it allows the variant GID4 protein to bind to a wider range of peptides having an N-terminal proline.

It may be provided that the variant GID4 protein exhibits a reduced influence from the identity of a residue at the third position of a target peptide when binding to an N-terminal proline residue as compared to a native GID4 protein. One advantage of a reduced influence from the identity of the third residue is that it allows the variant GID4 protein to bind to a wider range of peptides having an N-terminal proline.

It may be provided that the variant GID4 protein comprises an A252V mutation. The A252V mutation, wherein the alanine residue at position 252 of the GID4 protein is replaced with a valine residue, may reduce the influence of the second and third residues of the target peptide on the binding of the variant GID4 protein. One advantage of reducing the influence of the second and third residues is that it provides the variant GID4 with a higher binding affinity for N-terminal proline residues as compared to the native protein.

It may be provided that the variant GID4 protein comprises an S253T mutation. The S253T mutation, wherein the serine residue at position 253 of the GID4 protein is replaced with a threonine residue, increases binding to the target peptide. One advantage of this mutation is that it provides the variant GID4 with a higher binding affinity for N-terminal proline residues as compared to the native protein.

It may be provided that the variant GID4 protein comprises an S283F mutation. The S283F mutation, wherein the serine residue at position 283 of the GID4 protein is replaced with a phenylalanine residue, can enhance the binding of the GID4 protein to the target peptide. One advantage of enhancing binding is that it provides the variant GID4 with a higher binding affinity for N-terminal proline residues as compared to the native protein.

It may be provided that the variant GID4 protein comprises an A252V and an S253T mutation. This combination of mutations can result in an additive effect on binding to the target peptide. One advantage of an additive effect is that it provides the variant GID4 with a higher binding affinity for N-terminal proline residues as compared to the native protein.

It may be provided that the variant GID4 protein comprises an A252V and an S283F mutation. One advantage of this combination of mutations is that it provides the variant GID4 with a higher binding affinity for N-terminal proline residues as compared to the native protein and the S283F mutation can reduce the impact of the identity of the second and third residues of the peptide on binding.

It may be provided that the variant GID4 protein comprises an A252V, an S253T, and an S283F mutation. One advantage of this combination of mutations is that it provides the variant GID4 with a higher binding affinity for N-terminal proline residues as compared to the native protein and the A252V and the S283F mutation can reduce the impact of the identity of the second and third residues of the peptide on binding.

It may be provided that the GID4 protein is a human GID4 protein. One advantage of using a human GID4 protein is that it is less likely to be immunogenic in humans as compared to a GID4 protein from another species.

It may be provided that the GID4 protein is a truncated GID4 protein. A truncated protein can be understood as a protein that is missing a portion of its amino acid sequence. One advantage of using a truncated GID4 protein is that it may be easier to express and purify.

It may be provided that the truncated GID4 protein comprises residues 116-300 of the GID4 protein. One advantage of this particular truncated GID4 protein is that it retains its ability to bind to peptides with N-terminal proline.

It may be provided that the GID4 N-terminal proline binder further comprises a detectable label attached to the variant GID4 protein. A detectable label can be understood as a molecule that can be used to detect the presence of the variant GID4 protein. One advantage of including a detectable label is that it facilitates the detection of the binding of the variant GID4 protein to peptides having an N-terminal proline.

It may be provided that the GID4 N-terminal proline binder is formulated for use in protein sequencing. Protein sequencing can be understood as the process of determining the amino acid sequence of a protein. One advantage of formulating the GID4 N-terminal proline binder for use in protein sequencing is that it ensures that the GID4 N-terminal proline binder is compatible with other reagents used in protein sequencing.

One aspect of the present invention relates to a kit. A kit may be understood as a set of articles or implements used for a specific purpose. It may be provided that the kit comprises the GID4 N-terminal proline binder. The GID4 N-terminal proline binder may comprise a variant of a GID4 protein comprising at least one mutation selected from the group consisting of a substitution at position 252, position 253, and position 283 of SEQ ID NO: 1, wherein the variant GID4 protein exhibits an increased binding affinity for an N-terminal proline residue as compared to a native GID4 protein. One advantage of including the GID4 N-terminal proline binder in the kit is that it provides all of the necessary reagents for identifying N-terminal proline residues in peptides. The kit further comprises instructions for using the GID4 N-terminal proline binder in protein sequencing. Instructions for use may be understood as a description of how to use the GID4 N-terminal proline binder. One advantage of including instructions for use is that it facilitates the use of the GID4 N-terminal proline binder by providing a step-by-step guide.

One aspect of the present invention relates to a process for determining a sequence of a peptide. A peptide can be understood as a short chain of amino acids. It may be provided that the process comprises contacting the peptide with a variant GID4 protein. A GID4 protein can be understood as a protein that recognizes and binds to N-terminal proline residues in a peptide, which is particularly important for protein sequencing as proline residues cannot be chemically modified. The variant GID4 protein comprises at least one mutation selected from the group consisting of a substitution at position 252, position 253, and position 283 of SEQ ID NO: 1. One advantage of including a mutation selected from the group is that it provides the variant GID4 protein with a higher binding affinity for N-terminal proline residues as compared to the native protein. Moreover, the mutation can reduce the influence of the identity of the residue at the second and/or third position of the target peptide on the binding of the variant GID4 protein. The variant GID4 protein exhibits an increased binding affinity for an N-terminal proline residue as compared to a native GID4 protein. One advantage of an increased binding affinity is that it permits the variant GID4 protein to be more selective for peptides having an N-terminal proline. The process further comprises determining whether the variant GID4 protein binds to the peptide. Determining binding can be done by any method known in the art, such as surface plasmon resonance or bio-layer interferometry. One advantage of this step is that it allows for the identification of peptides that have an N-terminal proline. The process further comprises identifying the presence of an N-terminal proline in the peptide based on the binding of the variant GID4 protein to the peptide. One advantage of this step is that it allows for the determination of the sequence of the peptide.

It may be provided that the process further comprises removing the N-terminal proline residue of the peptide and repeating the steps of contacting, determining, and identifying. One advantage of this step is that it allows for the sequential determination of the amino acid sequence of the peptide.

It may be provided that the variant GID4 protein comprises an A252V mutation. The A252V mutation, wherein the alanine residue at position 252 of the GID4 protein is replaced with a valine residue, may reduce the influence of the second and third residues of the target peptide on the binding of the variant GID4 protein. One advantage of reducing the influence of the second and third residues is that it provides the variant GID4 with a higher binding affinity for N-terminal proline residues as compared to the native protein.

It may be provided that the variant GID4 protein comprises an S253T mutation. The S253T mutation, wherein the serine residue at position 253 of the GID4 protein is replaced with a threonine residue, increases binding to the target peptide. One advantage of this mutation is that it provides the variant GID4 with a higher binding affinity for N-terminal proline residues as compared to the native protein.

It may be provided that the variant GID4 protein comprises an S283F mutation. The S283F mutation, wherein the serine residue at position 283 of the GID4 protein is replaced with a phenylalanine residue, can enhance the binding of the GID4 protein to the target peptide. One advantage of enhancing binding is that it provides the variant GID4 with a higher binding affinity for N-terminal proline residues as compared to the native protein.

It may be provided that the variant GID4 protein comprises an A252V and an S253T mutation. This combination of mutations can result in an additive effect on binding to the target peptide. One advantage of an additive effect is that it provides the variant GID4 with a higher binding affinity for N-terminal proline residues as compared to the native protein.

It may be provided that the variant GID4 protein comprises an A252V and an S283F mutation. One advantage of this combination of mutations is that it provides the variant GID4 with a higher binding affinity for N-terminal proline residues as compared to the native protein and the S283F mutation can reduce the impact of the identity of the second and third residues of the peptide on binding.

It may be provided that the variant GID4 protein comprises an A252V, an S253T, and an S283F mutation. One advantage of this combination of mutations is that it provides the variant GID4 with a higher binding affinity for N-terminal proline residues as compared to the native protein and the A252V and the S283F mutation can reduce the impact of the identity of the second and third residues of the peptide on binding.

It may be provided that the variant GID4 protein is attached to a solid support. A solid support can be understood as a solid material to which the variant GID4 protein is attached. One advantage of attaching the variant GID4 protein to a solid support is that it facilitates the separation of the variant GID4 protein from the peptide after binding.

It may be provided that the variant GID4 protein is attached to a detectable label. A detectable label can be understood as a molecule that can be used to detect the presence of the variant GID4 protein. One advantage of including a detectable label is that it facilitates the detection of the binding of the variant GID4 protein to peptides having an N-terminal proline.

It may be provided that the GID4 protein is a human GID4 protein. One advantage of using a human GID4 protein is that it is less likely to be immunogenic in humans as compared to a GID4 protein from another species.

It may be provided that the GID4 protein is a truncated GID4 protein. A truncated protein can be understood as a protein that is missing a portion of its amino acid sequence. One advantage of using a truncated GID4 protein is that it may be easier to express and purify.

It may be provided that the truncated GID4 protein comprises residues 116-300 of the GID4 protein. One advantage of this particular truncated GID4 protein is that it retains its ability to bind to peptides with N-terminal proline.

It may be provided that the peptide is immobilized on a solid support. Immobilization of the peptide can be achieved by any method known in the art, such as covalent attachment or non-covalent binding. One advantage of immobilizing the peptide is that it facilitates the separation of the peptide from the variant GID4 protein after binding.

It may be provided that the peptide is part of a sample comprising a plurality of peptides. A sample of peptides can be a complex mixture, such as a cell lysate or a biological fluid. One advantage of using a sample comprising a plurality of peptides is that it allows for the identification of all peptides in the sample that have an N-terminal proline.

### BRIEF DESCRIPTION OF THE DRAWINGS

The following description cannot be considered limiting in any way. Various objectives, features, and advantages of the disclosed subject matter can be more fully appreciated with reference to the following detailed description of the disclosed subject matter when considered in connection with the following drawings, in which like reference numerals identify like elements.

FIG. 1 shows, according to some embodiments, directed evolution, yeast-surface display, selection, and sorting of GID4. GID4 was randomly mutagenized by (a) error-prone PCR and (b) inserted into pCTCON2 plasmid in EBY100 cells via homologous recombination. (c) The library was expressed by yeast-surface display and incubated with biotinylated peptide and fluorescent probes to detect expression of full-length protein (α-c-Myc antibody) and binding of peptide (SAPE). (d) The library was sorted by FACS and cells that fully expressed the GID4 protein and bound to peptide PGLVKSK(biotin) (cells in Q2) were gated and collected.

FIG. 2 shows, according to some embodiments, gating of the three rounds of sort and the summary of mutations detected by sequencing sorted libraries. (a) The distribution of cells in each round of sort. Areas of Q2 gated to collect cells from each round of sort highlighted. Peptide concentration was decreased from 10 μmol/L in the first round, to 1 μmol/L in the second round, and to 0.5 μmol/L in the third round. (b) Summary of NGS from sort 3i and 3ii that shows percentage of reads with no mutation, 1 mutation, 2 mutations, 3 mutations, or ≥4 mutations.

FIG. 3 shows, according to some embodiments, BLI response of wild-type GID4, three single mutant variants, two double mutant variants, and one triple mutation variant with PGLVKSK(biotin) peptide immobilized on streptavidin biosensors. Ten μmol/L of proteins were loaded in 1×PBS buffer with 0.1% BSA. The response curves are an average of six replicates.

FIG. 4 shows, according to some embodiments, selectivity of wild-type GID4 and variants to Nt-Pro. Steady-state BLI response of GID4 variants binding biosensors immobilized with PGLVKSK(biotin) peptide or XGLVKSK(biotin) peptide (where X is Arg, Asp, Val, Phe, Ser, or Met). Ten μmol/L of proteins were loaded in 0.1% BSA in 1×PBS. The bars show the average signal of replicates, which are presented as dots. Wild-type (solid gray) and variants A252V (blue), S253T (green), S283F (magenta), A252V-S253T-S283F (purple), A252V-S253T (light blue), and A252V-S283F (orange) are presented

FIG. 5 shows, according to some embodiments, effect of P2 of peptide on the binding of GID4 wild-type and variants. Steady state BLI response with PGLVKSK(biotin) or PXLVKSK(biotin) peptides (where X is Arg, Asp, Val, Phe, Ser, Met, or Pro). The bars show the average signal of replicates, which are presented as dots. Wild-type (solid gray) and variants A252V (blue), S253T (green), S283F (magenta), A252V-S253T-S283F (purple), A252V-S253T (light blue), and A252V-S283F (orange) are presented.

FIG. 6 shows, according to some embodiments, effect of P3 position of peptide on the binding of GID4 wild-type and variants. Steady-state BLI response with PGLVKSK(biotin) or PGXVKSK(biotin) peptides (where X is Arg, Asp, Val, Phe, Ser, Met, or Pro). The bars show the average signal of replicates, which are presented as dots. Wild-type (solid gray) and variants A252V (blue), S253T (green), S283F (magenta), A252V-S253T-S283F (purple), A252V-S253T (light blue), and A252V-S283F (orange) presented.

FIG. 7 shows, according to some embodiments, primers that were used for error-prone PCR or NGS sequencing.

FIG. 8 shows, according to some embodiments, average inflection point temperature (Ti) of GID4 wild-type and variants.

FIG. 9 shows, according to some embodiments, a distribution of the number of mutations in the naïve GID4 library.

FIG. 10 shows, according to some embodiments, a sequence of GID4 and PDB of GID4 bound to a hexapeptide (PDB:6CDG). (a) The full sequence of GID4 along with secondary structure features of truncated GID4 (116-300) indicated. Residues mutated in the top three enriched variants indicated with stars. (b) Structure of truncated GID4 (123-284) bound to PGLWKS peptide (yellow) with location of mutations of the top variants colored in blue (A252), green (A253), and magenta (S283).

FIG. 11 shows, according to some embodiments, data of BLI response curves from FIG. 4 with XGLVKSK(biotin) peptides. X is Arg, Asp, Val, Phe, Ser, Met, or Pro.

FIG. 12 shows, according to some embodiments, data of BLI response curves from FIG. 5 with PXLVKSK(biotin) peptides. X is Arg, Asp, Val, Phe, Ser, Met, or Pro.

FIG. 13 shows, according to some embodiments, data of BLI response curves from FIG. 6 with PGXVKSK(biotin) peptides. X is Arg, Asp, Val, Phe, Ser, Met, or Pro.

FIG. 14 shows, according to some embodiments, a sequence of a gene fragment used in implementing the study described in the Example.

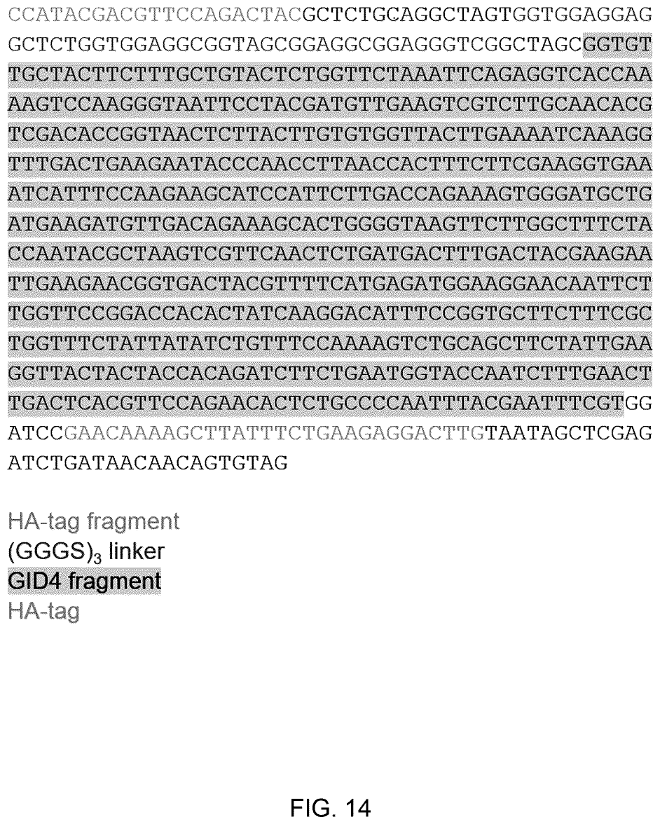

### DETAILED DESCRIPTION

A detailed description of one or more embodiments is presented herein by way of exemplification and not limitation.

Native GID4 protein has weak affinity to N-terminal proline, and the binding is heavily impacted by the identity of amino acids in the second and third position of the target peptide.

The GID4 N-terminal proline binder overcomes these deficiencies. It has been discovered that a GID4 N-terminal proline binder including a variant of a GID4 protein, the variant GID4 protein comprising at least one mutation selected from the group consisting of a substitution at position 252, position 253, and position 283 of SEQ ID NO: 1, exhibits an increased binding affinity for an N-terminal proline residue as compared to a native GID4 protein. One advantage of the GID4 N-terminal proline binder is that the increased binding affinity of the variant GID4 protein permits the GID4 N-terminal proline binder to be more selective for peptides having an N-terminal proline.

In an embodiment, a GID4 N-terminal proline binder, comprising: a variant of a GID4 protein (SEQ ID NO: 1) referred to as variant GID4 protein, the variant GID4 protein comprising at least one mutation selected from the group consisting of a substitution at position 252 of SEQ ID NO: 1, a substitution at position 253 of SEQ ID NO: 1, and a substitution at position 283 of SEQ ID NO: 1, wherein the variant GID4 protein exhibits an increased binding affinity for an N-terminal proline residue as compared to the native GID4 protein (SEQ ID NO: 1). If the mutation is only substitution at position 253 of SEQ ID NO: 1, the amino acid at position 253 is any amino acid except D and S. If the mutation is substitution at position 253 of SEQ ID NO: 1 in combination with substitution at position 252, position 283, or positions 252 and 283, the amino acid at position 253 is any amino acid except D. In an embodiment, the variant GID4 protein exhibits a reduced influence from the identity of a residue at the second position of a target peptide when binding to an N-terminal proline residue as compared to a native GID4 protein. In an embodiment, the variant GID4 protein exhibits a reduced influence from the identity of a residue at the third position of a target peptide when binding to an N-terminal proline residue as compared to a native GID4 protein. In an embodiment, the variant GID4 protein comprises an A252V mutation. In an embodiment, the variant GID4 protein comprises an S253T mutation. In an embodiment, the variant GID4 protein includes an S283F mutation. In an embodiment, the variant GID4 protein includes an A252V and an S253T mutation. In an embodiment, the variant GID4 protein includes an A252V and an S283F mutation. In an embodiment, the variant GID4 protein includes an A252V, an S253T, and an S283F mutation. In an embodiment, the GID4 protein is a human GID4 protein. In an embodiment, the GID4 protein is a truncated GID4 protein. In an embodiment, the truncated GID4 protein includes residues 116-300 of the GID4 protein. In an embodiment, the GID4 N-terminal proline binder includes a detectable label attached to the variant GID4 protein. In an embodiment, the GID4 N-terminal proline binder is formulated for use in protein sequencing.

The native GID4 protein has an amino acid sequence: MCARGQVGRGTQLRTGRPCSQVPGSRWRPERLLRRQRAGGRPSRPHPARARP GLSLPATLLGSRAAAAVPLPLPPALAPGDPAMPVRTECPPPAGASAASAASLIPPPP INTQQPGVATSLLYSGSKFRGHQKSKGNSYDVEVVLQHVDTGNSYLCGYLKIKGLT EEYPTLTTFFEGEIISKKHPFLTRKWDADEDVDRKHWGKFLAFYQYAKSFNSDDFD YEELKNGDYVFMRWKEQFLVPDHTIKDISGASFAGFYYICFQKSAASIEGYYYHRSS EWYQSLNLTHVPEHSAPIYEFR (SEQ ID NO:1).

In an embodiment, the variant GID4 protein has an amino acid sequence with a homology of at least 95% compared to that of: MCARGQVGRGTQLRTGRPCSQVPGSRWRPERLLRRQRAGGRPSRPHPARARP GLSLPATLLGSRAAAAVPLPLPPALAPGDPAMPVRTECPPPAGASAASAASLIPPPP INTQQPGVATSLLYSGSKFRGHQKSKGNSYDVEVVLQHVDTGNSYLCGYLKIKGLT EEYPTLTTFFEGEIISKKHPFLTRKWDADEDVDRKHWGKFLAFYQYAKSFNSDDFD YEELKNGDYVFMRWKEQFLVPDHTIKDISGX₁X₂FAGFYYICFQKSAASIEGYYYHRS SEWYQX₃LNLTHVPEHSAPIYEFR (SEQ ID NO:2), wherein X₂ is any amino acid except D or both D and S depending on the substitutions: if the mutation is only substitution at position 253 of SEQ ID NO: 1, the amino acid at position 253 is any amino acid except D and S; and if the mutation is substitution at position 253 of SEQ ID NO: 1 in combination with substitution at position 252, position 283, or positions 252 and 283, the amino acid at position 253 is any amino acid except D; and X₁ and X₃ are independently any amino acid.

In an embodiment, kit for determining a sequence of a peptide includes: the GID4 N-terminal proline binder and instructions for using the GID4 N-terminal proline binder in protein sequencing.

The GID4 N-terminal proline binder includes a variant of a GID4 protein. The GID4 protein is responsible for recognizing and binding to N-terminal proline residues in a peptide, facilitating the identification of proline during protein sequencing. The variant GID4 protein includes at least one mutation selected from the group consisting of a substitution at position 252 of SEQ ID NO: 1, a substitution at position 253 of SEQ ID NO: 1, and a substitution at position 283 of SEQ ID NO: 1, e.g., the group consisting of A252V, S253T, and S283F. These mutations enhance the binding affinity and selectivity of the GID4 protein for N-terminal proline residues while reducing the influence of the identity of the second and third residues of the target peptide on binding. The variant GID4 protein exhibits an increased binding affinity for an N-terminal proline residue as compared to a native GID4 protein, allowing for more specific and efficient detection of proline residues during protein sequencing.

The specific implementation of a variant GID4 protein in the GID4 N-terminal proline binder achieves several technical advantages, including increased binding affinity for N-terminal proline residues, reduced influence from neighboring residues, and enhanced selectivity for proline-containing peptides. These advantages contribute to the overall improvement of protein sequencing efficiency and accuracy.

The variant GID4 protein may exhibit a reduced influence from the identity of a residue at the second position of a target peptide when binding to an N-terminal proline residue as compared to a native GID4 protein, enhancing the versatility and applicability of the GID4 N-terminal proline binder in protein sequencing applications. Further, the variant GID4 protein may exhibit a reduced influence from the identity of a residue at the third position of a target peptide when binding to an N-terminal proline residue as compared to a native GID4 protein, further broadening the range of peptides that can be effectively recognized and bound.

The variant GID4 protein may include an A252V mutation, wherein the alanine residue at position 252 of the GID4 protein is replaced with a valine residue, leading to a more adaptable binding pocket that accommodates a wider range of amino acids at the second and third positions of the target peptide. The variant GID4 protein may comprise an S253T mutation, wherein the serine residue at position 253 of the GID4 protein is replaced with a threonine residue, resulting in improved binding interactions with the target peptide. Additionally, the variant GID4 protein may comprise an S283F mutation, wherein the serine residue at position 283 of the GID4 protein is replaced with a phenylalanine residue, further enhancing binding affinity and selectivity for N-terminal proline residues.

The variant GID4 protein may include a combination of mutations, such as A252V and S253T, leading to a synergistic effect on binding affinity and selectivity. The variant GID4 protein may comprise an A252V and an S283F mutation, combining the benefits of improved binding affinity and reduced influence from neighboring residues. Moreover, the variant GID4 protein may comprise an A252V, an S253T, and an S283F mutation, further enhancing the overall performance of the GID4 N-terminal proline binder in protein sequencing applications.

The GID4 protein may be a human GID4 protein, reducing the risk of immunogenicity and potential adverse reactions when used in human-related applications. The GID4 protein may be a truncated GID4 protein, wherein a portion of the amino acid sequence is removed, simplifying the production and purification processes.

The truncated GID4 protein may comprise residues 116-300 of the GID4 protein, retaining the essential binding region for N-terminal proline recognition while reducing the complexity of the protein structure. The GID4 N-terminal proline binder may further comprise a detectable label attached to the variant GID4 protein, facilitating the visualization and quantification of binding events during protein sequencing. Additionally, the GID4 N-terminal proline binder may be formulated for use in protein sequencing, ensuring compatibility with established protocols and procedures.

The present invention may also encompass a kit comprising the GID4 N-terminal proline binder and instructions for using the GID4 N-terminal proline binder in protein sequencing, providing a convenient and user-friendly package for researchers and practitioners in the field.

Each element of the embodiments contributes to specific technical advantages, including reduced influence from neighboring residues, increased binding affinity, improved selectivity, enhanced versatility, simplified production and purification, and compatibility with protein sequencing workflows. The combination of these advantages leads to a more efficient, accurate, and reliable method for identifying N-terminal proline residues in peptides, ultimately advancing the field of protein sequencing.

In an embodiment, a process for determining a sequence of a peptide comprising: contacting the peptide with a variant GID4 protein, the variant GID4 protein including at least one mutation selected from the group consisting of a substitution at position 252 of SEQ ID NO: 1, a substitution at position 253 of SEQ ID NO: 1, and a substitution at position 283 of SEQ ID NO: 1; and the variant GID4 protein exhibits an increased binding affinity for an N-terminal proline residue as compared to a native GID4 protein; determining whether the variant GID4 protein binds to the peptide; and identifying the presence of an N-terminal proline in the peptide based on the binding of the variant GID4 protein to the peptide if the mutation is only substitution at position 252 of SEQ ID NO: 1, the amino acid at position 253 is any amino acid except D and S; and if the mutation is substitution at position 253 of SEQ ID NO: 1 in combination with substitution at position 252, position 283, or positions 252 and 283, the amino acid at position 253 is any amino acid except D.

In an embodiment, the process includes removing the N-terminal proline residue of the peptide and repeating the preceding steps. In an embodiment, the variant GID4 protein includes an A252V mutation. In an embodiment, the variant GID4 protein includes an S253T mutation. In an embodiment, the variant GID4 protein includes an S283F mutation. In an embodiment, the variant GID4 protein includes an A252V and an S253T mutation. In an embodiment, the variant GID4 protein includes an A252V and an S283F mutation. In an embodiment, the variant GID4 protein includes an A252V, an S253T, and an S283F mutation. In an embodiment, the variant GID4 protein is attached to a solid support. In an embodiment, the variant GID4 protein is attached to a detectable label. In an embodiment, the GID4 protein is a human GID4 protein. In an embodiment, the GID4 protein is a truncated GID4 protein. In an embodiment, the truncated GID4 protein includes residues 116-300 of the GID4 protein. In an embodiment, the peptide is immobilized on a solid support. In an embodiment, the peptide is part of a sample including a plurality of peptides.

The process for determining a sequence of a peptide involves contacting the peptide with a variant GID4 protein. The GID4 protein plays a crucial role in recognizing and binding to N-terminal proline residues in peptides, which is particularly important in protein sequencing as proline cannot be readily chemically modified for detection. The variant GID4 protein used in this process contains at least one mutation selected from the group consisting of a substitution at position 252 of SEQ ID NO: 1, a substitution at position 253 of SEQ ID NO: 1, and a substitution at position 283 of SEQ ID NO: 1, e.g., the group consisting of A252V, S253T, and S283F. These mutations enhance the binding affinity and selectivity of the GID4 protein for N-terminal proline while minimizing the influence of neighboring residues. The variant GID4 protein exhibits an increased binding affinity for N-terminal proline residues as compared to the native protein, ensuring specific and efficient detection during sequencing.

The process further involves determining whether the variant GID4 protein binds to the peptide. This binding determination can be achieved using various methods known in the art, such as surface plasmon resonance or bio-layer interferometry. By assessing the binding interaction, the presence or absence of an N-terminal proline residue in the peptide can be established. The final step of the process involves identifying the presence of an N-terminal proline in the peptide based on the binding of the variant GID4 protein. If binding occurs, it indicates that the peptide has a proline residue at its N-terminus. This information is crucial for determining the sequence of the peptide and understanding its structure and function.

The process for determining a sequence of a peptide offers several technical advantages due to the specific implementation of each step. Contacting the peptide with a variant GID4 protein ensures selective binding to N-terminal proline residues. Determining binding allows for the identification of proline-containing peptides, and the subsequent identification step provides valuable information for protein sequencing. These advantages contribute to a more efficient and accurate approach for analyzing peptide sequences and understanding protein structure and function.

The process may further involve removing the N-terminal proline residue of the peptide and repeating the steps of contacting, determining, and identifying. This iterative approach enables the sequential determination of the amino acid sequence of the peptide, providing valuable insights into protein structure and function.

The variant GID4 protein may comprise an A252V mutation, wherein the alanine residue at position 252 of the GID4 protein is replaced with a valine residue, enhancing the adaptability of the binding pocket and allowing for the recognition of a wider range of peptides. The variant GID4 protein may comprise an S253T mutation, wherein the serine residue at position 253 of the GID4 protein is replaced with a threonine residue, improving binding interactions and enhancing the overall efficiency of the process. Additionally, the variant GID4 protein may comprise an S283F mutation, wherein the serine residue at position 283 of the GID4 protein is replaced with a phenylalanine residue, further contributing to increased binding affinity and selectivity for N-terminal proline.

The variant GID4 protein may include a combination of mutations, such as A252V and S253T, creating a synergistic effect that enhances binding affinity and selectivity. The variant GID4 protein may comprise an A252V and an S283F mutation, combining the advantages of improved binding and reduced influence from neighboring residues. Additionally, the variant GID4 protein may comprise an A252V, an S253T, and an S283F mutation, further optimizing the performance of the GID4 protein in the peptide sequencing process.

The variant GID4 protein may be attached to a solid support, such as a resin or a bead, facilitating the separation of the protein-peptide complex from the unbound peptides and simplifying downstream analysis. The variant GID4 protein may be attached to a detectable label, such as a fluorescent dye or an enzyme, enabling the visualization and quantification of binding events and providing a means for monitoring the sequencing process.

The GID4 protein used in the process may be a human GID4 protein, minimizing the risk of immunogenicity and ensuring compatibility with human-related applications. The GID4 protein may be a truncated GID4 protein, where a portion of the amino acid sequence is removed, simplifying the production and purification processes while retaining the essential binding region for N-terminal proline recognition. The truncated GID4 protein may comprise residues 116-300 of the GID4 protein, ensuring both functionality and ease of production.

The peptide may be immobilized on a solid support, such as a microarray or a membrane, facilitating high-throughput analysis and enabling parallel processing of multiple peptides. Additionally, the peptide may be part of a sample comprising a plurality of peptides, such as a cell lysate or a biological fluid, allowing for the comprehensive identification of proline-containing peptides within a complex mixture.

The process for determining a sequence of a peptide provides advantages that include the ability to perform sequential amino acid determination, enhanced binding affinity and selectivity due to specific mutations, improved separation and detection capabilities through solid supports and detectable labels, and compatibility with complex peptide samples. The combination of these elements and their associated advantages leads to a more robust, efficient, and versatile approach for protein sequencing and peptide analysis.

In an embodiment, a process for producing a variant GID4 protein including: providing a nucleic acid encoding a GID4 protein; introducing at least one mutation into the nucleic acid, the at least one mutation encoding a variant GID4 protein, the variant GID4 protein comprising at least one mutation selected from the group consisting of a substitution at position 252 of SEQ ID NO: 1, a substitution at position 253 of SEQ ID NO: 1, and a substitution at position 283 of SEQ ID NO: 1, e.g., the group consisting of A252V, S253T, and S283F; introducing the nucleic acid into a host cell; and culturing the host cell under conditions suitable for expression of the variant GID4 protein. In an embodiment, the at least one mutation is introduced by error-prone PCR. In an embodiment, the at least one mutation is introduced by site-directed mutagenesis. In an embodiment, the at least one mutation is introduced by homologous recombination. In an embodiment, the process includes isolating the variant GID4 protein from the host cell. In an embodiment, the variant GID4 protein includes an A252V mutation. In an embodiment, the variant GID4 protein includes an S253T mutation. In an embodiment, the variant GID4 protein includes an S283F mutation. In an embodiment, the GID4 protein is a human GID4 protein. In an embodiment, the GID4 protein is a truncated GID4 protein.

The process for producing a variant GID4 protein begins by providing a nucleic acid encoding a GID4 protein. The nucleic acid serves as the template for generating the variant GID4 protein with enhanced binding properties. The next step involves introducing at least one mutation into the nucleic acid. This mutation encodes a variant GID4 protein with at least one mutation selected from the group consisting of a substitution at position 252 of SEQ ID NO: 1, a substitution at position 253 of SEQ ID NO: 1, and a substitution at position 283 of SEQ ID NO: 1, e.g., the group consisting of A252V, S253T, and S283F. These mutations are specifically chosen to improve the binding affinity and selectivity of the GID4 protein for N-terminal proline residues while reducing the influence of neighboring residues.

The process then involves introducing the mutated nucleic acid into a host cell. The host cell serves as the environment for protein production. Culturing the host cell under conditions suitable for expression of the variant GID4 protein allows for the production of the engineered protein. These conditions may include providing necessary nutrients and maintaining optimal temperature and PH levels.

Technical advantages recap: The process for producing a variant GID4 protein ensures the efficient generation of the engineered protein with enhanced binding properties. Providing the nucleic acid encoding the GID4 protein establishes the foundation for protein production. Introducing mutations into the nucleic acid leads to the desired modifications in the protein sequence. Introducing the mutated nucleic acid into a host cell and culturing it under suitable conditions enables the expression and production of the variant GID4 protein with improved functionality for protein sequencing applications.

The at least one mutation may be introduced by error-prone PCR, which is a method for introducing random mutations into a DNA sequence. Alternatively, the at least one mutation may be introduced by site-directed mutagenesis. Site-directed mutagenesis is a method for introducing specific mutations into a DNA sequence. The at least one mutation may be introduced by homologous recombination, which is a method for exchanging DNA sequences between two DNA molecules.

The process may further comprise isolating the variant GID4 protein from the host cell. Isolating the variant GID4 protein can be achieved by any protein purification method known in the art, such as affinity chromatography or size-exclusion chromatography. The variant GID4 protein may comprise an A252V mutation, leading to a more adaptable binding pocket that can accommodate a wider range of amino acids at the second and third positions of the target peptide. The variant GID4 protein may comprise an S253T mutation, which can improve binding interactions with the target peptide. Additionally, the variant GID4 protein may comprise an S283F mutation, further enhancing binding affinity and selectivity for N-terminal proline residues.

The GID4 protein may be a human GID4 protein, reducing the risk of immunogenicity and ensuring compatibility with human-related applications. The GID4 protein may be a truncated GID4 protein, where a portion of the amino acid sequence is removed, simplifying the production and purification processes while maintaining the essential binding region for N-terminal proline recognition. The truncated GID4 protein may comprise residues 116-300 of the GID4 protein, striking a balance between functionality and ease of production.

The embodiments associated with the process for producing a variant GID4 protein provide additional flexibility and efficiency. Error-prone PCR, site-directed mutagenesis, and homologous recombination offer different approaches for introducing mutations, allowing for the generation of a diverse range of variant proteins. Isolating the variant GID4 protein ensures the purity and functionality of the final product. The specific mutations A252V, S253T, and S283F contribute to improved binding affinity and selectivity for N-terminal proline residues. The option of using a human GID4 protein minimizes immunogenicity concerns, and the use of a truncated GID4 protein simplifies production and purification processes. These elements work together to optimize the production of variant GID4 proteins with enhanced properties for protein sequencing applications.

It is contemplated that GID4 N-terminal proline binder 200 and determining a sequence of a peptide can include the properties, functionality, hardware, and process steps described herein and embodied in any of the following non-exhaustive list:
a process (e.g., a computer-implemented method including various steps; or a method carried out by a computer including various steps);
an apparatus, device, or system (e.g., a data processing apparatus, device, or system including means for carrying out such various steps of the process; a data processing apparatus, device, or system including means for carrying out various steps; a data processing apparatus, device, or system including a processor adapted to or configured to perform such various steps of the process);
a computer program product (e.g., a computer program product including instructions which, when the program is executed by a computer, cause the computer to carry out such various steps of the process; a computer program product including instructions which, when the program is executed by a computer, cause the computer to carry out various steps);
computer-readable storage medium or data carrier (e.g., a computer-readable storage medium including instructions which, when executed by a computer, cause the computer to carry out such various steps of the process; a computer-readable storage medium including instructions which, when executed by a computer, cause the computer to carry out various steps; a computer-readable data carrier having stored thereon the computer program product; a data carrier signal carrying the computer program product);
a computer program product including comprising instructions which, when the program is executed by a first computer, cause the first computer to encode data by performing certain steps and to transmit the encoded data to a second computer; or
a computer program product including instructions which, when the program is executed by a second computer, cause the second computer to receive encoded data from a first computer and decode the received data by performing certain steps.

It should be understood that the calculations may be performed by any suitable computer system. Data is entered into a computing system via any suitable type of user interface, and may be stored in memory, which may be any suitable type of computer readable and programmable memory and is preferably a non-transitory, computer readable storage medium. Calculations are performed by processor, which may be any suitable type of computer processor and may be displayed to the user on display, which may be any suitable type of computer display. The processor may be associated with, or incorporated into, any suitable type of computing device, for example, a personal computer or a programmable logic controller. The display, the processor, the memory, and any associated computer readable recording media are in communication with one another by any suitable type of data bus, as is well known in the art. Examples of computer-readable recording media include non-transitory storage media, a magnetic recording apparatus, an optical disk, a magneto-optical disk, and/or a semiconductor memory (for example, RAM, ROM, etc.). Examples of magnetic recording apparatus that may be used in addition to the memory, or in place of the memory, include a hard disk device (HDD), a flexible disk (FD), and a magnetic tape (MT). Examples of the optical disk include a DVD (Digital Versatile Disc), a DVD-RAM, a CD-ROM (Compact Disc-Read Only Memory), and a CD-R (Recordable)/RW. It should be understood that non-transitory computer-readable media include all computer-readable media except for a transitory, propagating signal.

The present invention offers a significant advancement in the field of protein sequencing by providing engineered GID4 variants with enhanced binding affinity and selectivity for N-terminal proline residues. The GID4 N-terminal proline binder and methods described herein enable efficient and accurate identification of proline during protein sequencing, addressing a critical gap in current technologies. The invention's ability to overcome limitations associated with native GID4 protein and its adaptability to various peptide sequences make it a valuable tool for researchers and practitioners in proteomics and related fields.

The articles and processes herein are illustrated further by the following Example, which is non-limiting.

### EXAMPLE

### Engineering GID4 for Use as an N-Terminal Proline Binder Via Directed Evolution

Nucleic acid sequencing technologies have gone through extraordinary advancements in the past several decades, significantly increasing throughput while reducing cost. To create similar advancement in proteomics, numerous approaches are being investigated to advance protein sequencing. One of the promising approaches uses N-terminal amino acid binders (NAABs), also referred to as recognizers, that selectively can identify amino acids at the N-terminus of a peptide. However, there are only a few engineered NAABs currently available that bind to specific amino acids and meet the requirements of a biotechnology reagent. Therefore, additional NAABs need to be identified and engineered to enable confident identification and ultimately de novo protein sequencing. To fill this gap, a human protein GID4 was engineered to create a NAAB for N-terminal proline (Nt-Pro). While native GID4 binds Nt-Pro, its binding is weak (mM) and greatly influenced by the identity of residues following the Nt-Pro. Through directed evolution, yeast-surface display, and fluorescence-activated cell sorting, we identified sequence variants of GID4 with increased binding affinity to Nt-Pro. Moreover, variants with an A252V mutation showed a reduced influence from residues in the second and third position of the target peptide when binding to Nt-Pro. The workflow outlined here is shown to be a viable strategy for engineering NAABs, even when starting from native Nt binding proteins whose binding is strongly impacted by the identity of residues following Nt-amino acid.

The development of next-generation DNA sequencing revolutionized genomic studies and biotechnology development by dramatically reducing the time and cost of DNA sequencing. Similar development for protein sequencing will have the same impact on proteomics. Currently, a broad effort is underway to develop new protein sequencing techniques using various next-generation approaches, including methods that use biological or solid-state nanopores, residue-specific fluorescent labels, DNA hybridization, and N-terminal (Nt-) amino acid binders (NAABs). Each approach has its own potential advantages as well as drawbacks, which have been outlined in the recent reviews on the different approaches being taken to realize a next-generation protein sequencing method.

Fluoro-sequencing approaches using NAABs have the benefit that they do not rely on whether an amino acid side-chain group is chemically modifiable, which is required for approaches based on conjugation of a fluorescent label. NAABs specifically bind to an amino acid only when it is at the N-terminus of a peptide sequence. Bioinformatic studies of the human proteome suggest that recognition of several amino acids, but not all 20, can be used to identify the majority of the proteome through protein fingerprinting. Thus, with a sufficient number of NAABs, proteins could be confidently identified and eventually a full suite of NAABs could enable de novo sequencing.

Proline is a critical amino acid to identify in protein sequencing. The residue composes nearly 6% of human proteome and plays an important role in structural conformation of a protein by introducing a bend in the polypeptide chain. However, the side-chain cannot be chemically modified to attach fluorescent label or dye. A NAAB for proline would therefore be a valuable addition to the current NAAB repertoire and a worthy target for NAAB development.

A candidate protein for proline-binding NAAB exists in the eukaryotic N-degron system which targets proteins for degradation by the proteasome. The system recognizes an Nt-signal sequence or “degron”. The GID4 protein is a part of the degradation machinery that recognizes the Nt-proline (Nt-Pro) and direct it for degradation. Based on structural studies, the depth and width of the binding pocket of GID4 results in the binding affinity and selectivity of the protein to not only be affected by the Nt-Pro, but also the residues that follow the Nt-Pro amino acid.

Direct evolution approaches were used to find sequence variants of human GID4 that not only have increased binding response to Nt-Pro but are also less impacted by the identity of residues that follow the Nt-Pro in the target peptide. Such variants of GID4 would be a valuable NAAB for protein sequencing and could also be used in other biotechnology applications that will benefit from identification of Nt-Pro.

### Methods

### Random Mutagenesis Library Generation

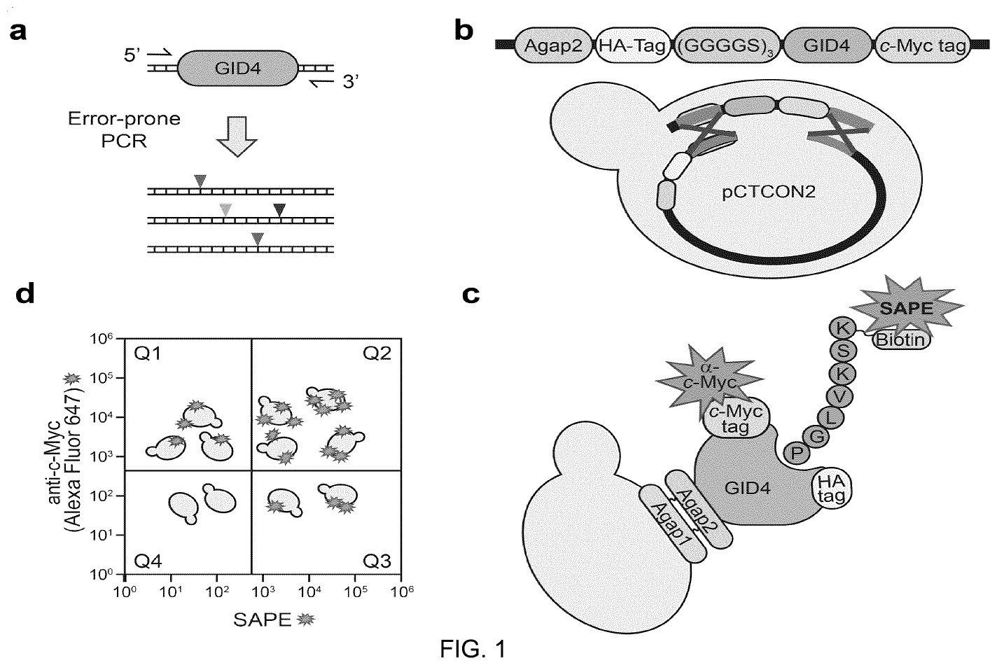

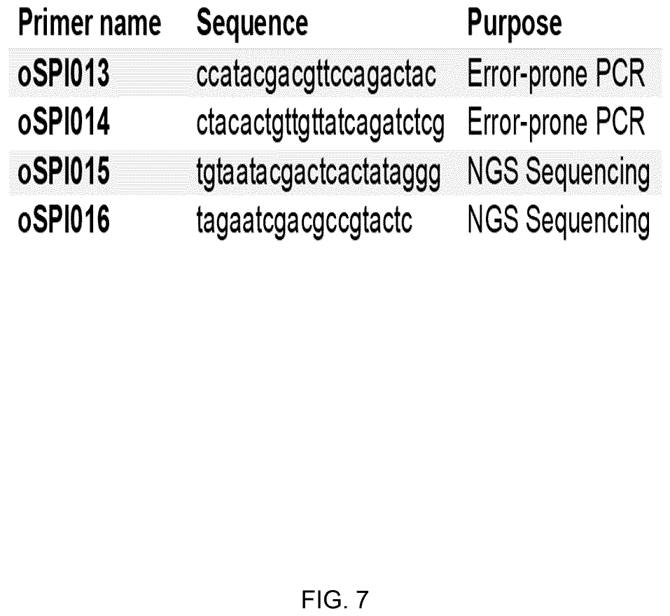

Random mutagenesis library of a GID4 fragment (residues 116-300, henceforth referred to as GID4) was generated using error-prone polymerase chain reaction (EP-PCR) (FIG. 1a). The gene fragment encoding for GID4 with a HA-tag (YPYDVPDYA) and (GGGGS)x3 linker at the N-terminus and a c-Myc-tag (EQKLISEEDL) at C-terminus was purchased from Twist Bioscience. The construct also included a NheI restriction site at the N-terminus and a BamHI at the C-terminus. The sequences at N-terminal and C-terminal ends of the gene fragment are homologous to sequence in the pCTCON2 plasmid. Six 50 μL EP-PCR aliquots of master mixture were prepared containing primers that bind to the gene fragment (oSPI013 and oSPI014, respectively, see FIG. 7), 0.05 U/μL of Taq polymerase, 1×Taq buffer (10 mmol/L Tris-HCl, 50 mmol/L KCl, 1.5 mmol/L MgCl₂, pH 8.3), 0.01 μg/μL bovine serum albumin (BSA), 3.95 mmol/L MgCl₂, 0.5 mmol/L MnCl₂, and unequal deoxynucleotide amounts of 0.23 mmol/L dATP, 0.2 mmol/L dCTP, 0.42 mmol/L dGTP, 2.9 mmol/L dTTP. The 0.5 fmol of template in each aliquot was amplified for 30 cycles, combined, and purified with QIAquick PCR Purification Kit (e.g., available commercially from Qiagen).

### Homologous Recombination in Yeast

The pCTCON2 plasmid was triple-digested with NheI-HF, BamH-HF, and SalI-HF restriction enzymes and purified with QIAquick PCR Purification Kit. Electrocompetent Saccharomyces cerevisiae EBY100 were prepared and transformed following procedures by Benatuil et al. Briefly, overnight culture of EBY100 in Yeast Extract-Peptone-Dextrose (YPD) medium was subcultured to optical density at 600 nm (OD₆₀₀)=0.2 in 100 mL of YPD and grown at 30° C. and 225 RPM to OD₆₀₀ of ˜1.6. Cells were pelleted and washed twice with 50 ml of ice-cold sterile water and once by ice-cold electroporation buffer (1 mol/L sorbitol with 1 mmol/L CaCl₂)). The washed cells were then suspended in 20 mL of 0.1 mol/L LiAc with 10 mmol/L dithiothreitol (DTT), and put in shaker-incubator for 30 min at 30° C., 225 RPM. Cells were pelleted, washed once in 50 mL of ice-cold electroporation buffer, and suspended to total volume of 1 mL with ice-cold electroporation buffer.

To transform the library (FIG. 1b), in 1.7 mL micro centrifuge tube on ice, 1 μg of linearized pCTCON2 vector, 4 μg of mutagenized GID4 insert, and 400 μL of electro competent cells were mixed and then transferred to 0.2 cm-gap electroporation cuvette on ice. After 5 min incubation, the cells were electroporated at 2.5 kV and 25 μF, and then transferred to 8 mL of recovery media (1:1 ratio of 1 mol/L sorbitol and YPD media) to recover for 1 h in a shaker-incubator at 30° C. and 225 RPM. The recovered cells were pelleted and suspended in 250 ml of SD-CAA media (20 g/L dextrose, 6.7 g/L Yeast Nitrogen Base, 5 g/L Yeast Synthetic Drop-out medium supplement without tryptophan, 5.4 g/L sodium phosphate dibasic, 8.56 g/L sodium phosphate monobasic monohydrate) with 10 μg/mL kanamycin (Kan). After overnight growth at 30° C. and 225 RPM, the cells were passaged to OD₆₀₀=0.1 in 1 L of fresh SD-CAA and grown at 30° C., 180 RPM in 4 L baffled flask for another 20 h. Cryogenic stocks of the library was prepared by concentrating the cells to OD₆₀₀ of ˜100 in 20% glycerol in SD-CAA media and aliquoting to 1 mL stocks.

### Library Expression, Screening, and Enrichment

A 1 mL cryogenic stock of the naïve library was thawed and suspended in 1 L of SD-CAA media with 2.5 μg/mL of Kan and carbenicillin (Carb) in 4 L baffled culture flask. After overnight growth at 30° C. and 180 RPM, the cells were subcultured to OD₆₀₀=1 in 25 mL of fresh SD-CAA media in 125 mL baffled flask. Cells were grown at 30° C., 225 RPM until they reached OD₆₀₀=3 and then subcultured to OD₆₀₀=1 in 25 mL of expression media composed of 10% SD-CAA and 90% SG-CAA (SG-CAA composed of 20 g/L galactose, 6.7 g/L Yeast Nitrogen Base, 5 g/L Yeast Synthetic Drop-out medium supplement without tryptophan, 5.4 g/L sodium phosphate dibasic, 8.56 g/L sodium phosphate monobasic monohydrate). Cells were expressed for 25 h at 20° C. and 225 RPM.

After expression, aliquots of cells were pelleted at 3000 RPM for 3 min and suspended to 1 mL for sort samples and 100 μL for control samples such that OD₆₀₀=1 in 0.1% BSA in 1× phosphate-buffered saline (PBS, 137 mmol/L NaCl, 2.7 mmol/L KCl, 10 mmol/L sodium phosphate dibasic, 1.8 mmol/L potassium phosphate monobasic, pH 7.4). The samples were prepared with or without 10 μmol/L of NH₂-PGLVKSK(biotin)-COOH peptide, which has biotin attached to the C-terminal lysine residue. Cells were incubated, while rotating with mixing for 90 min at room temperature. Cells were then pelleted at 3600 RPM for 3 min at 4° C. and suspended in 1 mL of 0.1% BSA in PBS with 10 μg/mL of α-c-Myc (9E1) antibody conjugated to Alexa Fluor® 647 and 25 μg/mL of streptavidin conjugated to R-phycoerythrin (FIG. 1c). For control samples, no or single fluorescent probe was included in 100 μL total volume. After incubation in the dark for 40 min on ice, the cells were washed with 0.1% BSA in PBS and kept on ice until running on cell sorter.

Cells were sorted by fluorescence-activated cell sorting (FACS) on Sony SH800 cell sorter with 100 μm chip. Control samples were used to set up compensation and gate for single cells that show binding to both fluorescent probes (Q2 in FIG. 1d). A total of 70345 cells were sorted into two 5 mL FACS tubes with 1 mL of SD-CAA in each and then combined in 14 mL Falcon cell culture tube for growth with additional 2 mL of SD-CAA media and 10 μg/mL of Carb. After ˜36 hrs of growth at 30° C. and 300 RPM, cells were subcultured to expression media and the selecting and sorting repeated, with modification of using 1 μmol/L of the peptide and collecting 10000 cells from gated areas. A third round of expression, selection, and sorting was done using 0.5 μmol/L of the biotinylated peptide. In each round of cell sorting and amplification, ˜10⁷ cells were pelleted and frozen for plasmid extraction.

### NGS Sample Prep and PacBio

The genes in the naïve library and the sorted pools of cells were sequenced using PacBio sequencing. The plasmids in the naïve and sorted library pools were extracted using Zymoprep Yeast Plasmid Miniprep II kit with modification of using QIAquick Spin Columns and eluting with 35 μL of nuclease-free water. Each sample was amplified with Q5 Hot Start High-Fidelity DNA Polymerase and primers oSPI015 and oSPI016. A master PCR mix of 150 μL was aliquoted to five 30 μL mixtures and amplified for 30 cycles. The aliquots were recombined and cleaned using QIAquick PCR Purification Kit and the concentration of amplicons were checked using a Qubit Fluorometer. Pacific Bioscience sequencing libraries were created for amplicons from the naïve library and sorted cells using SMRTBell Prep Kit 3.0 following the manufacturer's protocol for “Preparing multiplexed amplicon libraries using SMRTbell prep kit 3.0”. Libraries were barcoded during ligation of the SMRTbell adapter using the SMRTbell adapter index plate 96A. Following library construction, libraries were quantitated with a Qubit Fluorometer and sequenced for 10 h using the Pacific Biosciences Sequel binding kit 3.0 and Sequel Instrument. Following sequencing and primary analysis, a secondary analysis pipeline was developed to extract single molecule reads.

The de-barcoded PacBio HiFi CCS reads were generated by the SMRT Link (version 10.2.0.133434). The HiFi CCS reads were then aligned to the DNA reference sequence of the GID4 gene using minimap2 version 2.24 with the following parameters: -ax map-pb. Secondary alignments (with flag values of 256, 1024, or 2048) were excluded from subsequent analysis using samtools. Alignments containing insertions or deletions were also omitted from further analysis. Finally, the mapped sequences were translated into amino acids and compared to wild-type GID4 amino acid sequences for mutation analysis. Mutation profiles at both single molecular level and sample level were generated and plotted using custom Python scripts.

Expression and purification of wild-type and variants proteins

For expression and purification of the wild-type and variant GID4 proteins, the genes encoding for wild-type and top variants identified by the PacBio sequencing were cloned into the pET21(+) expression vector. Sequences encoding for six-histidine (His-tag) and a Tobacco Etch Virus (TEV) protease recognition site were included at the N-terminus of the GID4 genes.

For protein expression, BL21(DE3) cells were transformed with the plasmids encoding for the GID4 wild-type and variants. Cells were grown in 1 L of Luria-Bertani (LB) broth at 37° C., 225 RPM, to OD₆₀₀=0.5-0.8. The cells cultures were cooled to 16° C. and induced with 0.3 mmol/L of isopropyl β-D-1-thiogalactopyranoside (IPTG) for 20 h expression at 16° C. and 225 RPM. Cells were harvested by centrifugation and the cell pellets were stored at −80° C. until purification.

For purification, the frozen pellets were thawed and suspended in binding buffer (20 mmol/L Tris-HCl, 500 mmol/L NaCl, 10 mmol/L of imidazole, pH 7.4) and sonicated. The lysate was clarified by centrifuging at 20000×g for 30 min at 4° C. The cell lysate supernatant was loaded on a column with Sepharose 6 Fast Flow resin that was pre-charged with nickel. The resin was washed with 10× column volume (CV) of binding buffer, 5×CV of wash buffer 1 (20 mmol/L Tris-HCl, 500 mmol/L NaCl, 50 mmol/L imidazole, pH 7.4), 1.5×CV of wash buffer 2 (20 mmol/L Tris-HCl, 500 mmol/L NaCl, 100 mmol/L imidazole, pH 7.4). The proteins were eluted with 5×CV of elution buffer (20 mmol/L Tris-HCl, 500 mmol/L NaCl, 250 mmol/L imidazole, pH 7.4) and dialyzed to 1×PBS (pH 7.4) with 10K molecular-weight cut off (MWCO) Slide-A-Lyzer Dialysis Cassettes.

The purity of the samples were verified by running the products from the purifications on Any kD Mini-PROTEAN TGX Stain-Free Protein Gels, staining with Coomassie Blue R250 (1.25 g/L Coomassie Brilliant Blue R250, 45% methanol, and 10% acetic acid in water), and destaining with destain solution (40% methanol, and 10% acetic acid in water). The protein concentrations were determined using A280 measurements on Nanodrop 2000 spectrophotometer using extinction coefficient of 47330 (mol/L)⁻¹ cm⁻¹.

### Thermal Stability

The thermal stability of purified proteins was evaluated using Tycho NT.6. The instrument uses shift in the ratio of fluorescence signal from tryptophan and tyrosine at 350 nm and 330 nm from 35° C. to 95° C. to create thermal unfolding profile. The temperature at the inflection point (Ti) of the profile indicates the point at which protein shifts from folded to unfolded state. Ten UL of purified protein was loaded onto instrument-specific capillary tubes and ran in the instrument.

### Bio-Layer Interferometry (BLI)

High-throughput kinetic measurements were taken using Octet Red96e system with Octet Streptavidin (SA) Biosensors in 96-well format. The highest concentration of protein was prepared by mixing 25% of the total volume with 0.4% BSA in dialysis buffer (1×PBS) and completing subsequent dilutions with 0.1% BSA in dialysis buffer. The SA biosensors were hydrated for 10 min in the dialysis buffer with 0.1% BSA then loaded with NH₂-XXXVKSK(biotin)-COOH (where X are residues specified in the experiment and figures) by moving the sensors into wells with 10 μmol/L of the biotinylated peptide and incubating for 2 min. Unbound peptide was removed by incubating in the dialysis buffer with 0.1% BSA for 2 min in the next wells. The association of GID4 wild-type and variants were then measured by incubating the biosensors in wells with the proteins for 10 min and then dissociation measured by moving the sensors into new wells with dialysis buffer with 0.1% BSA for another 10 min. All wells contained 200 μL of specified solution. The measurements were analyzed with instrument software Data Analysis HT (Version 12).

### Directed Evolution of GID4

To identify variants of GID4 with increased binding for Nt-Pro, a directed evolution approach was used to introduce random mutations in the human GID4 sequence using error-prone PCR (EP-PCR) (FIG. 1a). It should be noted that the mutations are not completely random due to bias in EP-PCR from use of unequal amounts of deoxynucleotides. The full-length GID4 is poorly displayed on the yeast surface and therefore a truncated form of GID4 (residues 116-300) was used. Previous work showed that the truncated protein retain its ability to bind tetrapeptide with Nt-Pro as well as the full length protein.

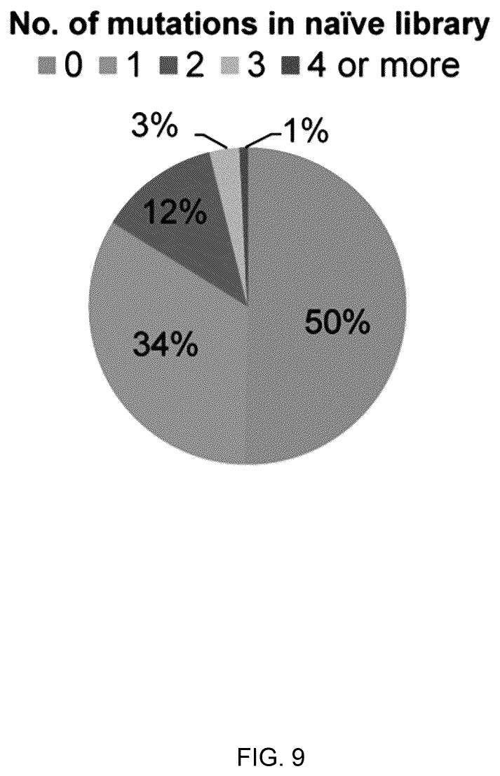

The randomly mutagenized GID4 sequences were inserted into pCTCON2 yeast-surface display plasmid and expressed in EBY100 S. cerevisiae cells. (FIG. 1b) To verify the diversity of the library, PacBio sequencing was utilized. The results showed that out of 22890 reads, 34% are single amino acid variant and 12% are double amino acid variants (FIG. 9).

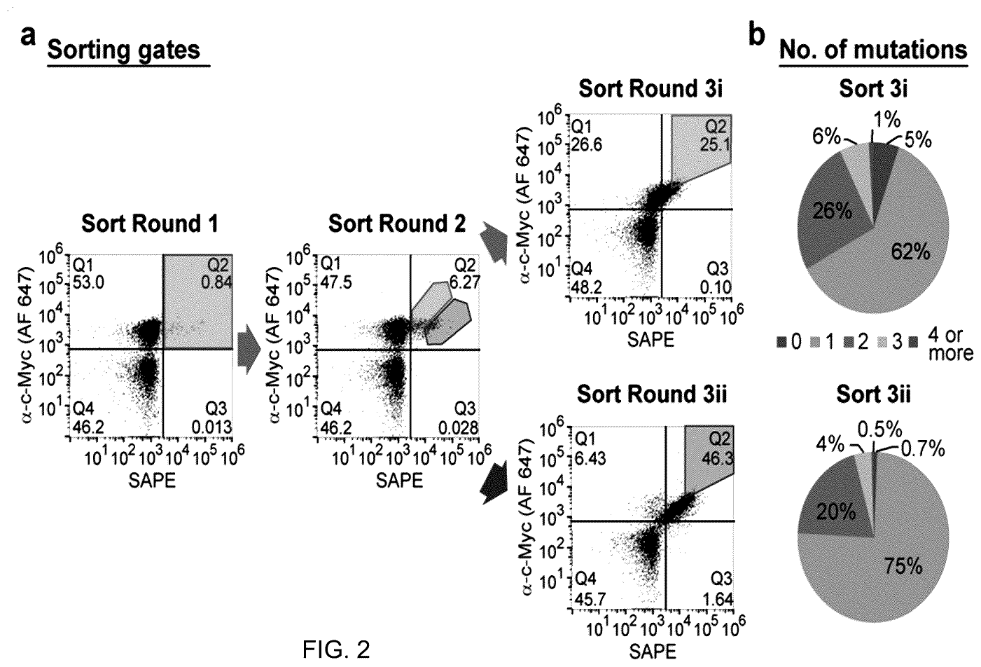

The naïve library went through three rounds of expression, selection, and amplification (FIG. 2a). The variants in the library that express full-length protein were selected by binding of fluorescently labeled α-c-Myc antibody to the c-Myc-tag fused at the C-terminus of GID4 (FIG. 1c). Binding to the target peptide PGLVKSK(biotin) was indirectly detected by binding of streptavidin conjugated with R-Phycoerythrin (SAPE) to the biotin that is conjugated to the bound target peptide (FIG. 1c). Cells that showed fluorescence from both binding events were collected (Q2 in FIG. 1d).

To select for variants with increased affinity for Nt-Pro, the amount of target peptide was decreased at each round of the three sorts, starting from 10 μmol/L and ending with 0.5 μmol/L. During the second round of sort using 1 μmol/L of peptide, two populations appeared to be present in Q2 (FIG. 2a). Thus, the two populates were gated separately, sorted, and grown. In the third round, both populations were expressed, selected, and enriched separately (Sort 3i and 3ii). PacBio NGS of Sort 3i and 3ii populations showed significant enrichment of single-point mutants, increasing from 34% of population in the naïve library (FIG. 9) to 61% and 75% in Sort 3i and 3ii, respectively. Sort 3ii (with 204629 full-length protein reads) was dominated by A252 to V (A252V) substitution (the residue position is with respect to the full length GID4 sequence), and it was the only mutation that had greater than 1% of the reads in the sort. Over 95% of the sequence reads contained A252V substitution. Out of those 95%, 77% were single substitution and the rest of the clones include 1-5 additional mutations. Sort 3i (with 204263 full-length protein reads) had a greater variety of mutations that were enriched. The top enriched variant is S238 to F (S283F) substitution present in 65% of the reads. Of those 65%, 47% of the reads are single mutant and the rest include a S283F in combination with 1 to 5 additional mutations. The S253T variant had the second highest % reads in Sort 3i at 14%.

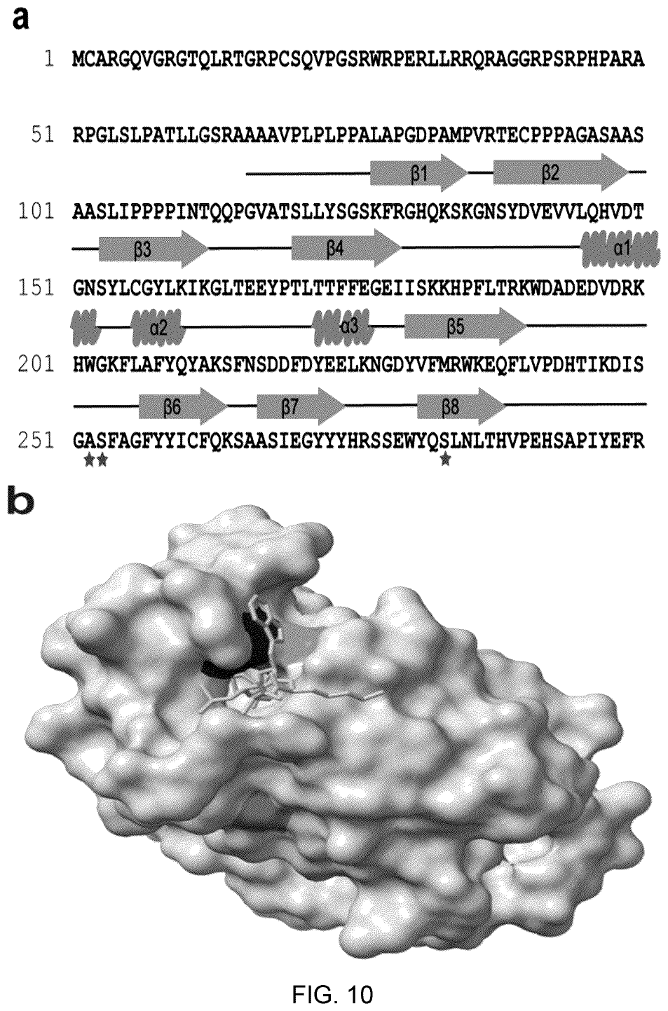

To identify the location of the top mutation sites with respect to structure of naïve GID4, the mutated residues were highlighted on human GID4 bound to PGLWKS peptide (PDB: 6CDG). A252 and S253 residues are part of the loops that form the binding pocket of GID4 (FIG. 10). S283, on the other hand, is part a B-strand that form the β-barrel of GID4.

### Thermal Stability of the Wild-Type and GID4 Variant Proteins

To compare the physical properties and binding properties of the top variants of GID4 identified by the screening, the wild-type and variant proteins were expressed and purified in E. coli. Three enriched variants were selected: the S283F and S253T from Sort 3i and the A252V from Sort 3ii. A triple mutant variant with all three mutations, A252V-S253T-S283F, and double mutant variants that contain A252V mutation, A252V-S253T and A252V-S283F, were also prepared. The protein sequences included an N-terminal His-tag and were express and purified from E. coli on a nickel-column.

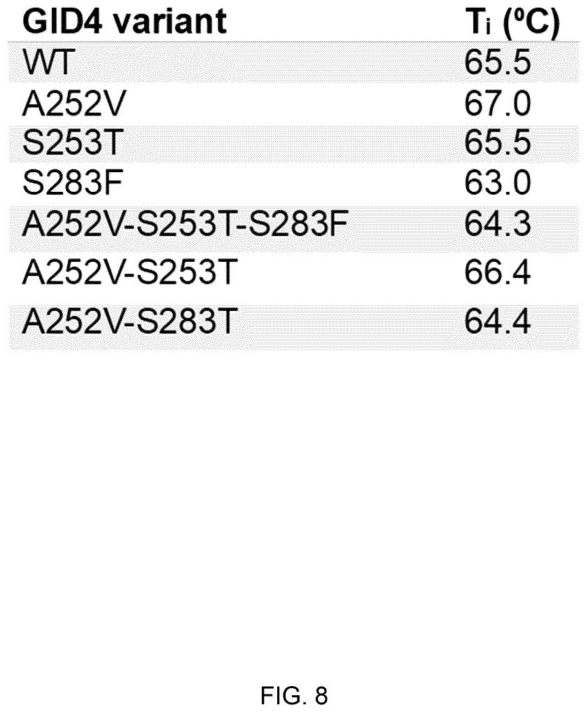

The effect of the substitutions on the thermal stability of the proteins was evaluated by measuring the thermal unfolding profiles, on Tycho NT.6, to determine the inflection point temperature (Ti). At Ti, the protein transitions from folded to unfolded state due to temperature increase. Shift in Ti can indicate change in thermal stability of the protein. The S253T variant showed no shift in Ti compared to the wild-type but A252V variant shows >1° C. increase, suggesting a slight increase in thermal stability (FIG. 8). S283F variant, on the other hand, shows >2° C. decrease in Ti, suggesting small decrease in stability. The triple and double mutants showed decrease in Ti with presence of S283F mutation. With presence of A252V mutation, Ti increases in A252V-S253T double mutant and appear to lessen the decrease in A252V-S283F mutant due to the presence of S283F mutation.

### GID4 Sequence Variants Bind to Nt-Pro Peptide

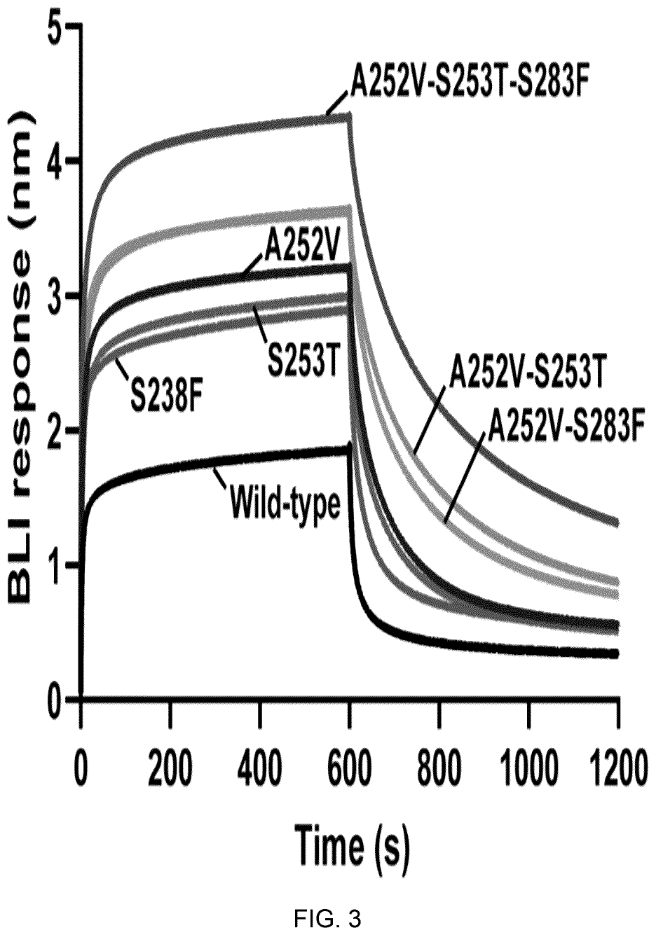

The association and dissociation of the GID4 wild-type and the six variants to the PGLVKSK(biotin) peptide were evaluated using BLI. BLI is an optical technique that can measure interaction between protein in solution ad peptide immobilized on the biosensor tip. Single point mutation variants A252V, S253T, and S283F all show a steady state response higher than the wild-type, with the A252V variant showing response >1.7× higher than the wild-type (FIG. 3). The S283F variant has an apparent faster dissociation rate compared to the other singe mutation variants. The apparent faster dissociation rate due to S283F mutation is also present in A252V-S283F mutant, when compared to the A252V-S253T mutant. The triple mutant A252V-S253T-S283F shows additive effect of the mutations. The triple mutant's steady state response is >2.3× higher than the wild-type and it has slower apparent dissociation rate than the wild-type and other variants. The increase in the BLI response and shift in dissociation rate can be beneficial as an Nt-Pro recognizing protein; differences in off-rate can be used as method to distinguish different Nt-amino acids.

### GID4 Sequence Variants Maintain the Selectivity to Nt-Pro

The FACS analysis and sorting demonstrated that the three GID4 variants bind to the peptide PGLVKSK(biotin). However, the assay does not provide information on the specificity of the variant proteins. To determine the specificities of the variants, the binding of the variant proteins to peptides with different Nt-amino acids was analyzed using BLI.

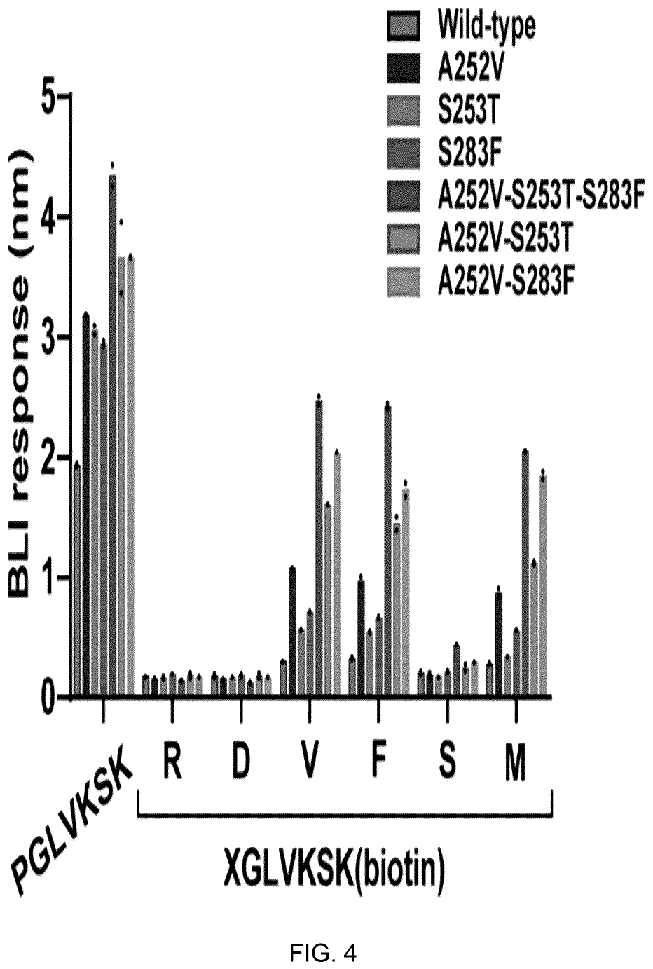

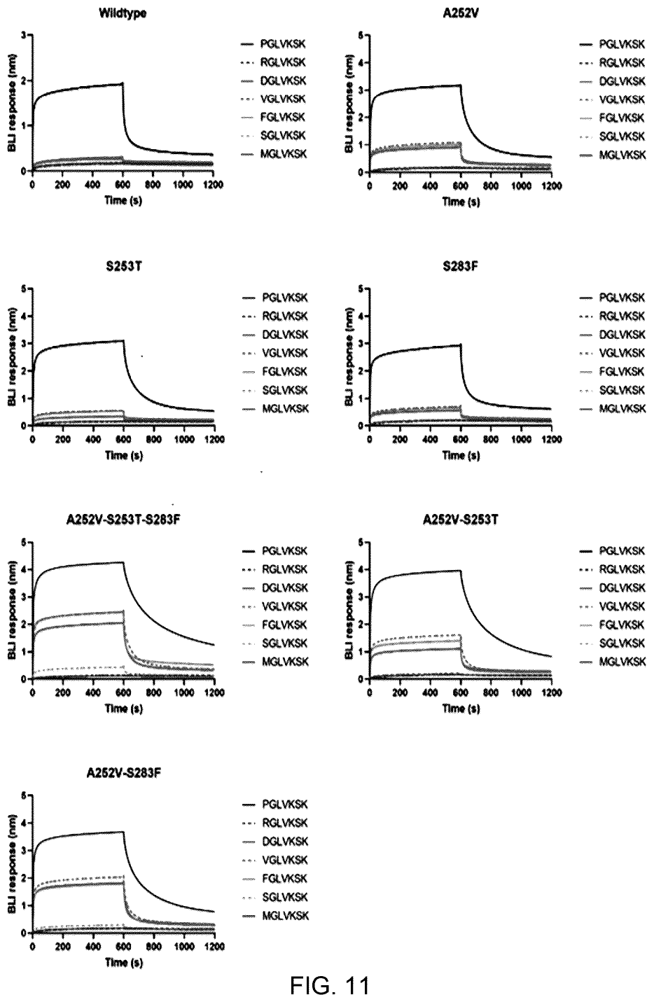

The BLI responses of the wild-type and variant proteins were evaluated using seven peptides derived from peptide used in the FACS, XGLVKSK(biotin), where the Nt-Pro at X is replaced by Nt-Arg (R), Asp (D), Val (V), Phe (F), Ser(S), or Met (M). These Nt-amino acids represent residues with different properties, including aromatic, positive and negatively charged, and hydrophobic. To simplify the presentation of the data, FIG. 4 shows the steady state BLI response values for each GID4 protein variant binding to each of the seven peptides. Representative association and dissociation curves are in FIG. 3. Overall, all six variants maintain selectivity for Nt-Pro, showing higher BLI response with Nt-Pro compared to the other tested amino acids. While binding to peptides to Nt-Val, Phe, and Met is also observed, as previously reported, these response levels are still lower than with Nt-Pro. The variants also have slower apparent off-rate with Nt-Pro than with Nt-Val, Phe, or Met, which will further help distinguish the binding to Nt-Pro than to the rest of the residues (FIG. 11).

### A252V Mutation Reduces Impact of P2 on Nt-Pro Binding

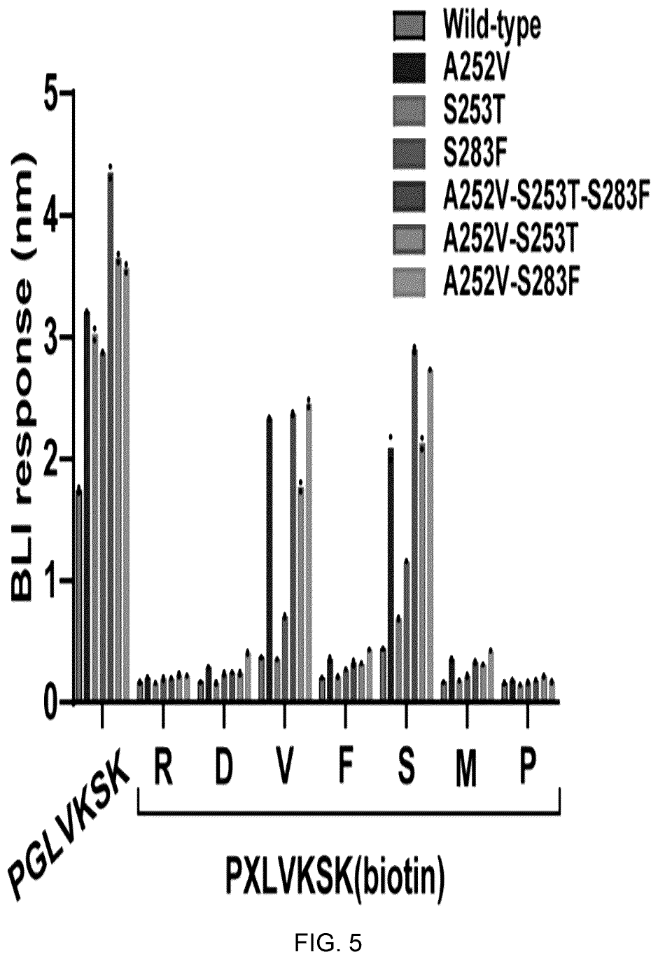

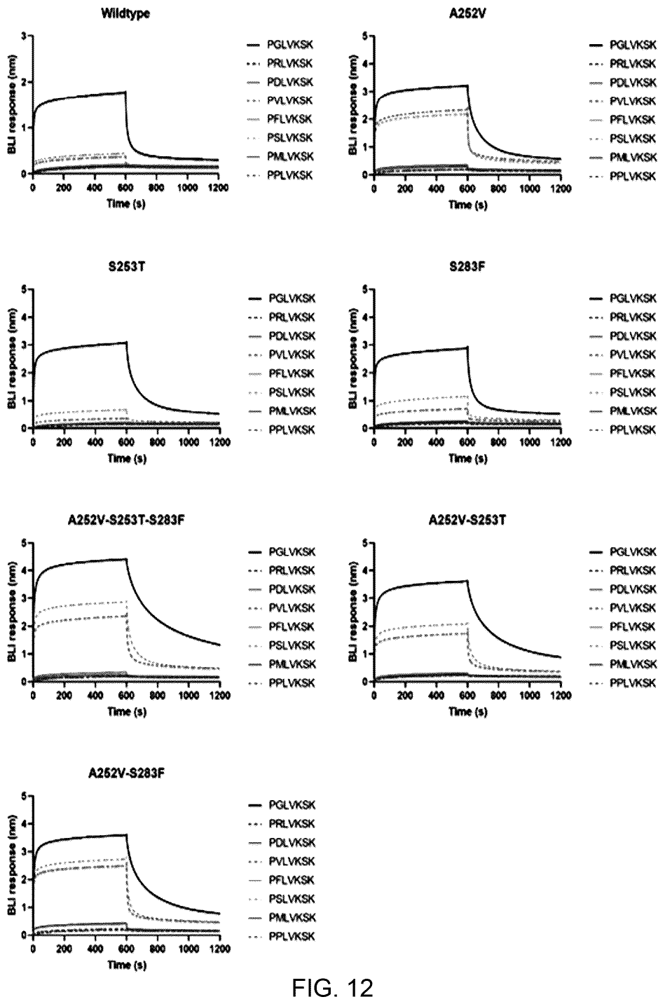

Previous works on GID4, as well as other N-degron proteins reported significant impact of the second position of the peptide (P2) on binding affinity. The impact of the P2 on the top variants were evaluated with BLI assay using PGLVKSK(biotin) and PXLVKSK(biotin) peptides, where X is Arg, Asp, Val, Phe, Ser, Met, or Pro. In agreement with previous works, the steady state BLI response level of wild-type is significantly reduced when the Gly in P2 of the peptide is replaced by one of the seven tested amino acids (FIG. 5 and FIG. 12). S283F variant and, especially, A252V variant retain higher level of BLI response compared to wild-type when Val or Ser is at P2. S283F variant retains 40% of signal with Ser at P2 and A252V variant retains >65% with Val and Ser. This retention of binding response with Val and Ser at P2, relative to Gly at P2, is maintained with the triple mutant the double mutants, with over 58% of signal retained for these variants when Ser is at P2. The A252V-S283F mutant and the triple mutant retained >55% of signal when Val is at P2.

GID4 sequence variants reduce the impact of P3 on Nt-Pro binding.

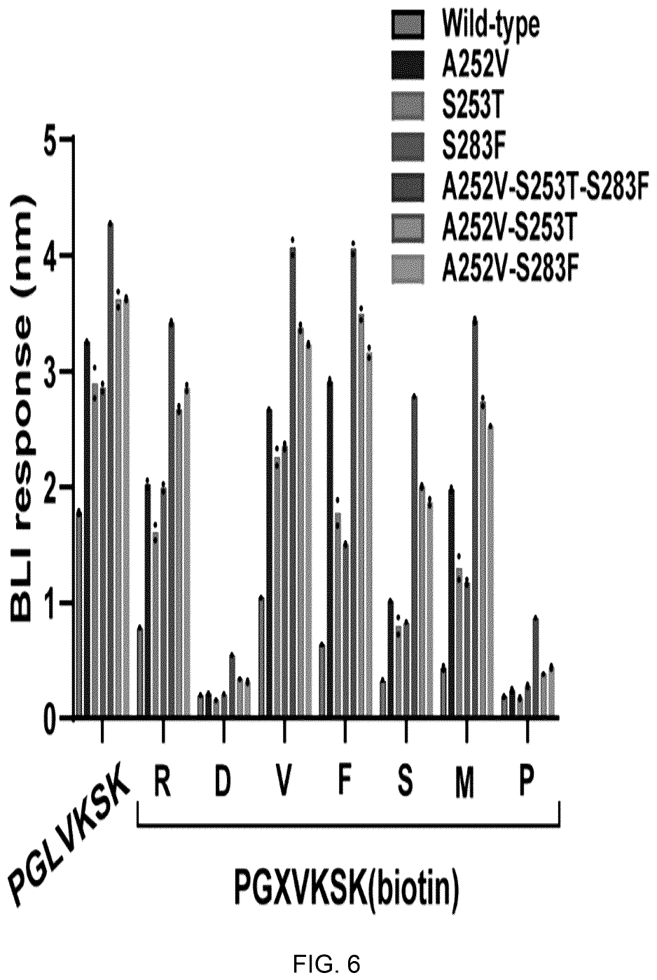

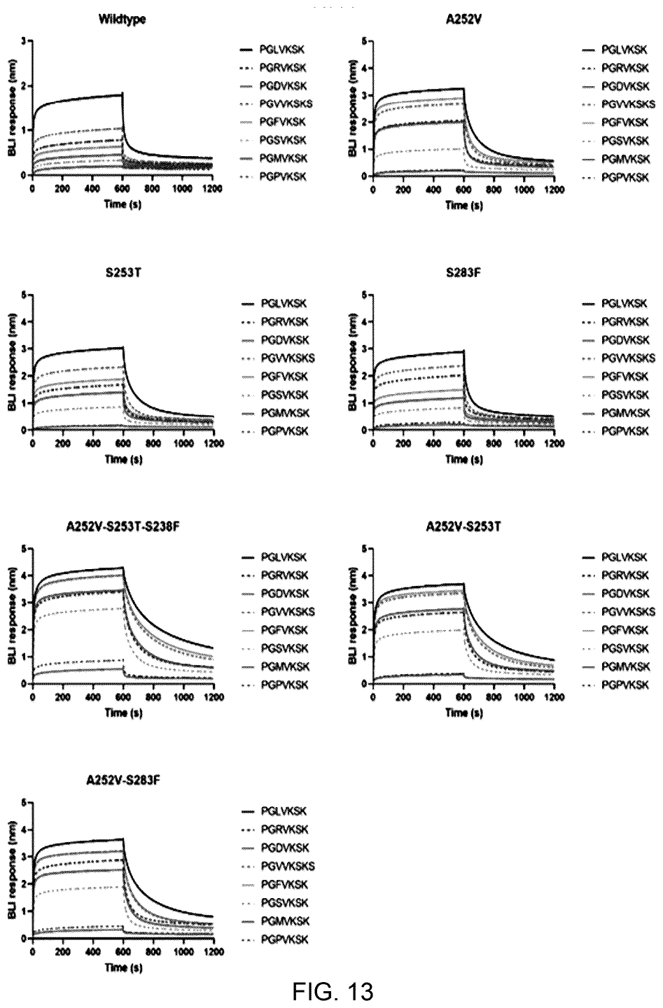

Due to the depth of the binding pocket of GID4, the binding is impacted by the identity of residues in the target peptide beyond P2. As other works showed less impact from P4 and beyond, this study focused on impact of the P3 position. Similar to evaluating the impact of P2, the impact of P3 was evaluated via BLI with PGLVKSK(biotin) peptide and PGXVKSK(biotin) peptides, where X is Arg, Asp, Val, Phe, Ser, Met, or Pro. The reduction in steady state BLI response of wild-type with PGXVKSK(biotin) peptides reaffirm that binding is significantly impacted by identify of residue at P3 (FIG. 6 and FIG. 13). Asp and Pro are particularly not favorable at P3, as almost no BLI signal was measured. The BLI response of variants with the rest of the peptides, however, increased. The A252V variant shows >60% of BLI response with X=Arg, Val, Phe, or Met relative to the response with PGLVKSK(biotin) (FIG. 6). The double mutant variants and the triple mutant variants retain even greater response, with >69% of BLI response retained with X=Arg, Val, Phe, or Met and >51% with X=Ser, relative to the response with PGLVKSK(biotin). Wild-type GID4, in comparison, showed relative BLI signal >50% only with Val at P3. These results suggest that the tested variants of GID4 not only increased overall signal of binding to Nt-Pro, but also mitigate some of the impact of the identity of the P3 position of the binding target peptide.

As part of broad effort to develop next-generation protein sequencing technologies, NAAB proteins have been successfully engineered to bind with increased affinity and selectivity to charged, aromatic and hydrophobic residues that include Phe and Leu. This study aimed to engineer the human GID4 protein as a NAAB for Nt-Pro by enhancing its affinity and specificity to Nt-Pro using directed evolution approach. Three GID4 mutations that increase binding, in comparison to the wild-type protein were identified: A252V, S253T, and S283F.

The involvement of S253 position was not surprising as it was previously identified as playing a role in binding Pro in the wild-type GID4. While S253D mutation was reported to decrease binding, we found the S253T mutation to increase binding response. The shift in position of the hydroxyl group in the side-chain of Ser to Thr could affect hydrogen-bond formation within the binding pocket that may better accommodate the target peptide. The A252 and S283 positions were not previously identified to play a role in binding to target peptide. The S283F variant was an unexpected find as S283 is part of the β-barrel in the wild-type human GID4, which is positioned away from the binding pocket (FIG. 10b). The mutation from the small polar side-chain of Ser to the bulky hydrophobic side-chain of Phe may have led to a conformational change that decreases the thermal stability but enhances binding to the peptide.

The A252V mutation is particularly exciting as the mutation not only increases the Nt-Pro binding and thermal stability of the protein (FIG. 8), but it can accommodate residues with greater side-chain volume in the P2 position of target peptides (FIG. 5). This effect is maintained in the triple mutant A252V-S253T-S283F and the double mutants A252V-S253T and A252V-S283F. However, combining the S253T mutation with A252V mutation appears to decrease the effect of A252V mutation for P2 position. The steady state BLI responses with A252V-S253T variant are less than A252V or other multiple mutation variants (FIG. 5). As A252 and S253 are next to each other, their mutations are likely to affect one another. The identity of P2 impacting binding affinity of target peptide is not unique to GID4. Other N-degron proteins such as bacterial ClpS from the Leu/N-degron pathway and eukaryotic UBR box from Arg/N-degron pathway are also affected by P2. Furthermore, due to the depth and width of the GID4 binding pocket, binding is impacted by identity of residues beyond P2 in the target peptide. Fortunately, all six variants showed increase in binding response with other tested residues at P3, except for Asp and Pro. Reduction in binding due Asp and Pro at P3 is consistent with previous works. A252V shows the most increase in tolerance of different amino acids at P3 among the three single mutants (FIG. 6). The variant retained higher binding response with Phe and Met at P3 compared the other two variants. The triple and double mutants showed even more increase in tolerance of different amino acids at P3. These variants retained >51% of binding response, relative to PGLVKSK(biotin) peptide, with Ser at P3 compared to <31% for single mutants, which suggest synergistic effect from the combination of mutations (FIG. 6).

This study successfully found variants of GID4 with increased binding to Nt-Pro, including the A252V variant and the A252V-S253T-S283F variant, which can accommodate greater variety of residues at P2 and P3 in target peptide without diminishing binding. These results suggest that additional engineering of the GID4 could be aimed at further decreasing the effect of P2 and P3 positions of the peptide on binding by selecting for variants with binding pocket that could accommodate even greater variety in amino acid residues at positions P2 and P3. Such GID4 variants impacted even less by the identity of the P2 and P3 amino acid of target peptide would further increase the potential use as an Nt-Pro NAAB and in other biotechnology applications.

The processes described herein may be embodied in, and fully automated via, software code modules executed by a computing system that includes one or more general purpose computers or processors. The code modules may be stored in any type of non-transitory computer-readable medium or other computer storage device. Some or all the methods may alternatively be embodied in specialized computer hardware. In addition, the components referred to herein may be implemented in hardware, software, firmware, or a combination thereof.

Many other variations than those described herein will be apparent from this disclosure. For example, depending on the embodiment, certain acts, events, or functions of any of the algorithms described herein can be performed in a different sequence, can be added, merged, or left out altogether (e.g., not all described acts or events are necessary for the practice of the algorithms). Moreover, in certain embodiments, acts or events can be performed concurrently, e.g., through multi-threaded processing, interrupt processing, or multiple processors or processor cores or on other parallel architectures, rather than sequentially. In addition, different tasks or processes can be performed by different machines and/or computing systems that can function together.

Any logical blocks, modules, and algorithm elements described or used in connection with the embodiments disclosed herein can be implemented as electronic hardware, computer software, or combinations of both. To clearly illustrate this interchangeability of hardware and software, various illustrative components, blocks, modules, and elements have been described above generally in terms of their functionality. Whether such functionality is implemented as hardware or software depends upon the particular application and design constraints imposed on the overall system. The described functionality can be implemented in varying ways for each particular application, but such implementation decisions should not be interpreted as causing a departure from the scope of the disclosure.

The various illustrative logical blocks and modules described or used in connection with the embodiments disclosed herein can be implemented or performed by a machine, such as a processing unit or processor, a digital signal processor (DSP), an application specific integrated circuit (ASIC), a field programmable gate array (FPGA) or other programmable logic device, discrete gate or transistor logic, discrete hardware components, or any combination thereof designed to perform the functions described herein. A processor can be a microprocessor, but in the alternative, the processor can be a controller, microcontroller, or state machine, combinations of the same, or the like. A processor can include electrical circuitry configured to process computer-executable instructions. In another embodiment, a processor includes an FPGA or other programmable device that performs logic operations without processing computer-executable instructions. A processor can also be implemented as a combination of computing devices, e.g., a combination of a DSP and a microprocessor, a plurality of microprocessors, one or more microprocessors in conjunction with a DSP core, or any other such configuration. Although described herein primarily with respect to digital technology, a processor may also include primarily analog components. For example, some or all of the signal processing algorithms described herein may be implemented in analog circuitry or mixed analog and digital circuitry. A computing environment can include any type of computer system, including, but not limited to, a computer system based on a microprocessor, a mainframe computer, a digital signal processor, a portable computing device, a device controller, or a computational engine within an appliance, to name a few.

The elements of a method, process, or algorithm described in connection with the embodiments disclosed herein can be embodied directly in hardware, in a software module stored in one or more memory devices and executed by one or more processors, or in a combination of the two. A software module can reside in RAM memory, flash memory, ROM memory, EPROM memory, EEPROM memory, registers, hard disk, a removable disk, a CD-ROM, or any other form of non-transitory computer-readable storage medium, media, or physical computer storage known in the art. An example storage medium can be coupled to the processor such that the processor can read information from, and write information to, the storage medium. In the alternative, the storage medium can be integral to the processor. The storage medium can be volatile or nonvolatile.

While one or more embodiments have been shown and described, modifications and substitutions may be made thereto without departing from the spirit and scope of the invention. Accordingly, it is to be understood that the present invention has been described by way of illustrations and not limitation. Embodiments herein can be used independently or can be combined.

All ranges disclosed herein are inclusive of the endpoints, and the endpoints are independently combinable with each other. The ranges are continuous and thus contain every value and subset thereof in the range. Unless otherwise stated or contextually inapplicable, all percentages, when expressing a quantity, are weight percentages. The suffix(s) as used herein is intended to include both the singular and the plural of the term that it modifies, thereby including at least one of that term (e.g., the colorant(s) includes at least one colorants). Option, optional, or optionally means that the subsequently described event or circumstance can or cannot occur, and that the description includes instances where the event occurs and instances where it does not. As used herein, combination is inclusive of blends, mixtures, alloys, reaction products, collection of elements, and the like.

As used herein, a combination thereof refers to a combination comprising at least one of the named constituents, components, compounds, or elements, optionally together with one or more of the same class of constituents, components, compounds, or elements.

All references are incorporated herein by reference.

The use of the terms “a,” “an,” and “the” and similar referents in the context of describing the invention (especially in the context of the following claims and embodiments) are to be construed to cover both the singular and the plural, unless otherwise indicated herein or clearly contradicted by context. It can further be noted that the terms first, second, primary, secondary, and the like herein do not denote any order, quantity, or importance, but rather are used to distinguish one element from another. It will also be understood that, although the terms first, second, etc. are, in some instances, used herein to describe various elements, these elements should not be limited by these terms. For example, a first current could be termed a second current, and, similarly, a second current could be termed a first current, without departing from the scope of the various described embodiments. The first current and the second current are both currents, but they are not the same condition unless explicitly stated as such.

The modifier about used in connection with a quantity is inclusive of the stated value and has the meaning dictated by the context (e.g., it includes the degree of error associated with measurement of the particular quantity). The conjunction or is used to link objects of a list or alternatives and is not disjunctive; rather the elements can be used separately or can be combined together under appropriate circumstances.

### PARTS LIST

GID4 N-terminal proline binder 200
gene fragment 201
HA-tag fragment 202
(GGGGS)₃ linker 203
GID4 fragment 204
HA-tag 205
   determining a sequence of a peptide//determines a sequence of a peptide

What is claimed is:

1. A GID4 N-terminal proline binder, comprising: a variant of a GID4 protein (SEQ ID NO: 1) referred to as variant GID4 protein, the variant GID4 protein comprising at least one mutation selected from the group consisting of a substitution at position 252 of SEQ ID NO: 1, a substitution at position 253 of SEQ ID NO: 1, and a substitution at position 283 of SEQ ID NO: 1; and the variant GID4 protein exhibits an increased binding affinity for an N-terminal proline residue as compared to native GID4 protein (SEQ ID NO: 1); and:
if the mutation is only substitution at position 253 of SEQ ID NO: 1, the amino acid at position 253 is any amino acid except D and S; and
if the mutation is substitution at position 253 of SEQ ID NO: 1 in combination with substitution at position 252, position 283, or positions 252 and 283, the amino acid at position 253 is any amino acid except D.

2. The GID4 N-terminal proline binder of claim 1, wherein the variant GID4 protein has an amino acid sequence with a homology of at least 95% compared to that of:
MCARGQVGRGTQLRTGRPCSQVPGSRWRPERLLRRQRAGGRPSRPHPARARP GLSLPATLLGSRAAAAVPLPLPPALAPGDPAMPVRTECPPPAGASAASAASLIPPPP INTQQPGVATSLLYSGSKFRGHQKSKGNSYDVEVVLQHVDTGNSYLCGYLKIKGLT EEYPTLTTFFEGEIISKKHPFLTRKWDADEDVDRKHWGKFLAFYQYAKSFNSDDFD YEELKNGDYVFMRWKEQFLVPDHTIKDISGX₁X₂FAGFYYICFQKSAASIEGYYYHRS SEWYQX₃LNLTHVPEHSAPIYEFR (SEQ ID NO:2),
wherein X₂ is any amino acid except D or both D and S depending on the substitutions recited in claim 1; and X₁ and X₃ are independently any amino acid.

3. The GID4 N-terminal proline binder of claim 1, wherein the variant GID4 protein exhibits a reduced influence from the identity of a residue at the second position of a target peptide when binding to an N-terminal proline residue as compared to a native GID4 protein.

4. The GID4 N-terminal proline binder of claim 1, wherein the variant GID4 protein exhibits a reduced influence from the identity of a residue at the third position of a target peptide when binding to an N-terminal proline residue as compared to a native GID4 protein.

5. The GID4 N-terminal proline binder of claim 1, wherein the variant GID4 protein comprises an A252V mutation.

6. The GID4 N-terminal proline binder of claim 1, wherein the variant GID4 protein comprises an S253T mutation.

7. The GID4 N-terminal proline binder of claim 1, wherein the variant GID4 protein comprises an S283F mutation.

8. The GID4 N-terminal proline binder of claim 1, wherein the variant GID4 protein comprises an A252V and an S253T mutation.

9. The GID4 N-terminal proline binder of claim 1, wherein the variant GID4 protein comprises an A252V and an S283F mutation.

10. The GID4 N-terminal proline binder of claim 1, wherein the variant GID4 protein comprises an A252V, an S253T, and an S283F mutation.

11. The GID4 N-terminal proline binder of claim 1, wherein the GID4 protein is a human GID4 protein.

12. The GID4 N-terminal proline binder of claim 1, wherein the GID4 protein is a truncated GID4 protein.

13. The GID4 N-terminal proline binder of claim 12, wherein the truncated GID4 protein comprises residues 116-300 of the GID4 protein.

14. The GID4 N-terminal proline binder of claim 1, wherein the GID4 N-terminal proline binder further comprises a detectable label attached to the variant GID4 protein.

15. The GID4 N-terminal proline binder of claim 1, wherein the GID4 N-terminal proline binder is formulated for use in protein sequencing.

16. A kit comprising: the GID4 N-terminal proline binder of claim 1 and instructions for using the GID4 N-terminal proline binder in protein sequencing.

17. A process for determining a sequence of a peptide comprising: contacting the peptide with a variant GID4 protein, the variant GID4 protein comprising at least one mutation selected from the group consisting of a substitution at position 252 of SEQ ID NO: 1, a substitution at position 253 of SEQ ID NO: 1, and a substitution at position 283 of SEQ ID NO: 1; and the variant GID4 protein exhibits an increased binding affinity for an N-terminal proline residue as compared to a native GID4 protein (SEQ ID NO: 1); determining whether the variant GID4 protein binds to the peptide; and identifying the presence of an N-terminal proline in the peptide based on the binding of the variant GID4 protein to the peptide; and
if the mutation is only substitution at position 253 of SEQ ID NO: 1, the amino acid at position 252 is any amino acid except D and S; and
if the mutation is substitution at position 253 of SEQ ID NO: 1 in combination with substitution at position 252, position 283, or positions 252 and 283, the amino acid at position 253 is any amino acid except D.

18. The process of claim 17, further comprising removing the N-terminal proline residue of the peptide and repeating the proceeding steps of the process.

19. The process of claim 17, wherein the variant GID4 protein comprises an A252V mutation.

20. The process of claim 17, wherein the variant GID4 protein comprises an S253T mutation.

21. The process of claim 17, wherein the variant GID4 protein comprises an S283F mutation.

22. The process of claim 17, wherein the variant GID4 protein comprises an A252V and an S253T mutation.

23. The process of claim 17, wherein the variant GID4 protein comprises an A252V and an S283F mutation.

24. The process of claim 17, wherein the variant GID4 protein comprises an A252V, an S253T, and an S283F mutation.

25. The process of claim 17, wherein the variant GID4 protein is attached to a solid support.

26. The process of claim 17, wherein the variant GID4 protein is attached to a detectable label.

27. The process of claim 17, wherein the GID4 protein is a human GID4 protein.

28. The process of claim 17, wherein the GID4 protein is a truncated GID4 protein.

29. The process of claim 28, wherein the truncated GID4 protein comprises residues 116-300 of the GID4 protein.

30. The process of claim 17, wherein the peptide is immobilized on a solid support.

31. The process of claim 17, wherein the peptide is part of a sample comprising a plurality of peptides.
